(19) United States
(12) Patent Application Publication
(10) Pub. No.: US 2024/0165026 A1
(43) Pub. Date: May 23, 2024

(54) PROTONATED pH RESPONSIVE POLYMER ENCAPSULATION OF BISPECIFIC ANTIBODIES AND CYTOKINES

(71) Applicant: OncoNano Medicine, Inc., Southlake, TX (US)

(72) Inventors: Tian Zhao, Allen, TX (US); Stephen Gutowski, Irving, TX (US); Irina Kalashnikova, Dallas, TX (US); Qingtai Su, Allen, TX (US); Jason Miller, Dallas, TX (US); Gaurav Bharadwaj, Issaquah, WA (US); Bhargavi Allu, Irving, TX (US); Austin Burcham, Southlake, TX (US); Zirong Chen, Prosper, TX (US); Ruolan Han, Houston, TX (US)

(21) Appl. No.: 18/502,783

(22) Filed: Nov. 6, 2023

Related U.S. Application Data
(60) Provisional application No. 63/382,528, filed on Nov. 6, 2022.
(60) Provisional application No. 63/459,351, filed on Apr. 14, 2023.

(51) Int. Cl. A61K 9/107; A61K 38/20; A61K 47/32; C07K 16/28; C07K 16/30; C07K 16/32

(52) U.S. Cl. CPC A61K 9/1075; A61K 38/20; A61K 38/2013; A61K 38/208; A61K 47/32; C07K 16/2803; C07K 16/2809; C07K 16/2887; C07K 16/30; C07K 16/32; A61K 2039/505

(57) ABSTRACT

Described herein are therapeutic pH responsive micelle compositions comprising a block copolymer and a biological therapeutic agent useful for the treatment of cancer, methods of making such micelles and intermediate compositions useful for making such micelles.

### BACKGROUND OF THE INVENTION

Multifunctional nanoparticles have received attention in a wide range of applications such as biosensors, diagnostic nanoprobes and targeted drug delivery systems. These efforts have been driven to a large extent by the need to improve biological specificity with reduced side effects in diagnosis and therapy through the precise, spatiotemporal control of agent delivery in various physiological systems. In order to achieve this goal, efforts have been dedicated to develop stimuli-responsive nanoplatforms. Environmental stimuli that have been exploited for pinpointing the delivery efficiency include pH, temperature, enzymatic expression, redox reaction and light induction. Among these activating signals, pH trigger is one of the most extensively studied stimuli based on two types of pH differences: (a) pathological (e.g. tumor) vs. normal tissues and (b) acidic intracellular compartments.

For example, due to the unusual acidity of the tumor extracellular microenvironment (pH ˜6.5), several pH-responsive nano systems have been reported to increase the sensitivity of tumor imaging or the efficacy of therapy. However, for polymer micelle compositions that release drug by hydrolysis in acidic environments, it can take days for the release of the drug. In that time period, the body can excrete or break down the micelles.

To target the acidic endo-/lysosomal compartments, nanovectors with pH-cleavable linkers have been investigated to improve payload bioavailability. Furthermore, several smart nanovectors with pH-induced charge conversion have been designed to increase drug efficacy. The endocytic system is comprised of a series of compartments that have distinctive roles in the sorting, processing and degradation of internalized cargo. Selective targeting of different endocytic compartments by pH-sensitive nanoparticles is particularly challenging due to the short nanoparticle residence times (<mins) and small pH differences in these compartments (e.g. <1 pH unit between early endosomes and lysosomes. Ultra pH sensitive (UPS) nanoparticles remain as intact micelles at physiological pH (7.4) during blood circulation but disassembles when the environmental pH is reduced below the micelle transition pH (pHt) upon exposure to tumor acidic milieu.

Bispecific antibodies (BsAbs) are an important class of therapeutics for immune-oncology applications. T cell engagers (TCEs) target tumor associated antigens and T cells to eradicate antigen-expressing tumor cells. TCEs for solid tumors have likewise demonstrated encouraging clinical efficacy but shown dose-limiting toxicities due to on-target/off-tumor effects. For instance, patients receiving solitomab (EpCAM X CD3 bispecific) experienced severe gastrointestinal toxicity which precluded its further development.

Cytokines (e.g., IL-12, IL-2) and cytokine fusion proteins (e.g., IL-12Fc, IL-2Fc) can induce anti-tumor immune responses, but their clinical applications are limited by the unfavorable pharmacokinetic properties and serious dose-limiting toxicities (e.g., cytokine release syndrome, vascular leak syndrome, etc).

U.S. Pat. No. 9,751,970 to Jinming Gao et al., entitled “Block Copolymer and Micelle Compositions and Methods of Use Thereof” was granted on Sep. 5, 2017, and describes micelle forming polymers that can based to entrap a therapeutic agent, including a chemotherapy agent. The '970 patent provides an example of encapsulation of doxorubicin using PEO-b-PC6A. The method of the '970 patent includes dissolving doxorubicin and PEO-b-PC6A in water and hydrochloric acid. The solution is then added drop by drop into a 0.1M pH 9 buffer solution under sonication. Although the encapsulation method disclosed in the '970 patent is capable of encapsulating small molecules and some biological agents, the present inventors have found the method was incapable of encapsulating certain biomolecules, particularly cytokines and antibodies.

There remains a need in the art to provide a pH sensitive targeted delivery method of bispecific antibodies and cytokines to the tumor microenvironment.

### SUMMARY OF THE INVENTION

Polymer encapsulants, or micelles, described herein are therapeutic agents useful for the treatment of primary and metastatic tumor tissue (including lymph nodes). The block copolymers and micelle compositions presented herein exploit this ubiquitous pH difference between cancerous tissue and normal tissue and provides a highly sensitive and specific response after encountering the acidic pH of the tumor microenvironment, thus, allowing the deployment of a therapeutic payload to tumor tissues. The pH sensitive encapsulation minimizes off-tumor effects while providing targeted delivery to the acidic tumor environment. Encapsulation of a therapeutic payload is achieved by an acid protonated polymer intermediate to exhibit an in vitro pH-dependent activation window. The protonation of the polymer generates a strong positive charge on a region of the polymer. The positively charged region of the polymer attracts a negatively charged region in the therapeutic payload. The electrostatic interaction between the positively charged polymer and negatively charged therapeutic payload creates a physical approximation between the polymer chains and the biomolecule. Neutralization of the polymer and therapeutic payload results in a sudden increase of hydrophobicity of the positively charged polymer section which interacts with the hydrophobic regions in the therapeutic payload to form a stable encapsulated structure or micelle.

In some embodiments, the block copolymer of Formula (I) comprises poly(ethylene oxide) (PEO), and a hydrophobic polymer segment with the following structure:

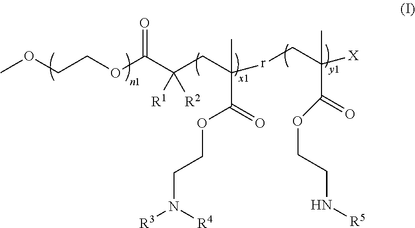

wherein: n₁ is an integer from 40-500, x₁ is an integer from 4-250, y₁ is an integer from 0-10, X is a halogen, —OH, or —C(O)OH, R¹ and R² are each independently hydrogen or optionally substituted C₁-C₆ alkyl, R³ and R⁴ are each independently an optionally substituted C₁-C₆ alkyl, C₃-C₁₀ cycloalkyl or aryl, or R³ and R⁴ are taken together with the corresponding nitrogen to which they are attached form an optionally substituted 5 to 7-membered ring, R⁵ is hydrogen or —C(O)CH₃. 5. In some embodiments, n₁ is an integer from 100-250, x₁ is an integer from 40-250, and/or y₁ is 0. In some embodiments, n₁ is an integer from 100-250, x₁ is an integer from 100-200, and/or y₁ is 0. In some embodiments, n₁ is an integer of about 114, x₁ is about 170, and/or y₁ is 0. In some embodiments, n₁ is an integer of 114, x₁ is 170, and/or y₁ is 0. The units of x1 may include the same units having the same R³ and R⁴ substituents or different units which have different R³ and R⁴ substituents. Further, the addition of other hydrophobic monomeric units in small quantities that do not significantly affect the ability of the micelle to encapsulate and release a biomolecule composition should be understood to be included in Formula (I).

In some embodiments, the hydrophobic polymer segment of the block copolymer of formula (I) is selected from:

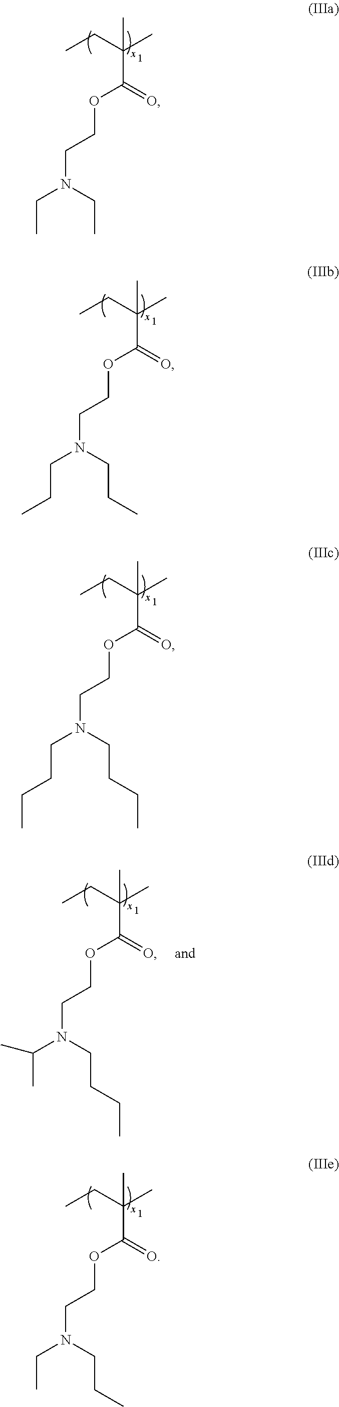

In some embodiments, the therapeutic agent is a biomolecule. In some embodiments, the therapeutic agent is a protein. In some embodiments, the therapeutic agent is a bispecific antibody (BsAbs). In some embodiments the therapeutic agent is a cytokine. In some embodiments the therapeutic agent is a cytokine fusion protein. In some embodiments the therapeutic agent is a human IL-12. In some embodiments the therapeutic agent is single chain human IL-12. In some embodiments the therapeutic agent is monovalent human IL-12 fused to the Fc region of the IgG antibody. In some embodiments the therapeutic agent is bivalent human IL-12 fused to the Fc region of the IgG antibody. In some embodiments the therapeutic agent is human IL-2. In some embodiments the therapeutic agent is bivalent human IL-2 fused to the Fc region of the IgG antibody. In some embodiments the therapeutic agent is a human IL-18. In some embodiments the therapeutic agent is a solitomab bispecific antibody T cell engager (TCE). In some embodiments the therapeutic agent is a runimotamab bispecific antibody T cell engager. In some embodiments the therapeutic agent is a blinatumomab bispecific antibody T cell engager. In some embodiments the therapeutic agent is glofitamab bispecific antibody T cell engager. In some embodiments the therapeutic agent is an odronextamab bispecific antibody T cell engager.

In some embodiments, the micelle has a diameter of less than about 1 μm or less than about 50 nm. In some embodiments, the micelle has diameter of about 25 to about 50 nm. In some embodiments, the micelle has diameter of about 20 to about 40 nm. In some embodiments, the micelle has a diameter of about 50 to about 70 nm.

In another aspect of the invention is a pH responsive composition comprising one or more micelles described herein. In some embodiments, the pH responsive composition has a pH transition point. In some embodiments, the pH transition point is between 4-8, 6-7.5, or 4.5-6.5. In some embodiments, composition has a pH response of less than 0.25 or 0.15 pH units.

In another aspect of the invention is a method for treating cancer in an individual in need thereof, comprising administration of an effective amount of a pH-sensitive micelle composition comprising a chemotherapeutic agent as described herein. In some embodiments, the cancer comprises a solid tumor. In some embodiments, the tumor is of a cancer, wherein the cancer is of the breast, ovarian, prostate, peritoneal metastasis, colorectal, bladder, esophageal, head and neck (HNSSC), lung, brain, kidney, renal, or skin (including melanoma and sarcoma). In some embodiments, the tumor is reduced in size by about 50%, about 60%, about 70%, about 80%, about 90%, or about 95%. In some embodiments, micelle described here is administered with one of more additional therapies. In some embodiments, the additional therapy is a checkpoint inhibitor. In some embodiments, the checkpoint inhibitor is an anti-PD-1 therapy, anti-PD-L1 therapy, or anti-CTLA-4 therapy.

Other objects, features and advantages of the block copolymers, micelle compositions, and methods described herein will become apparent from the following detailed description. It should be understood, however, that the detailed description and the specific examples, while indicating specific embodiments, are given by way of illustration only, since various changes and modifications within the spirit and scope of the instant disclosure will become apparent to those skilled in the art from this detailed description.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIG. 1 displays a scheme showing bispecific antibody (BsAb) encapsulation and tumor delivery with reduced systemic exposure.

FIG. 2A shows encapsulation of monovalent mouse IL-12-Fc and human IL-2Fc with a protonated polymer intermediate and pH-dependent activation in vitro displaying a large activation window by a reporter cell assay.

FIG. 2B shows encapsulation of monovalent mouse IL-12-Fc and human IL-2Fc without a protonated polymer intermediate and pH-dependent activation in vitro displaying a small or nonexistent activation window by a reporter cell assay.

FIG. 3A shows encapsulation of human IL-12 with a protonated polymer intermediate of PEG-PDBA₆₀ and pH-dependent activation in vitro by a reporter cell assay.

FIG. 3B shows encapsulation of human IL-12 with a protonated polymer intermediate of PEG-PDBA₁₇₀ and pH-dependent activation in vitro by a reporter cell assay.

FIG. 4A shows encapsulation of single chain human IL-12 with a protonated polymer intermediate of PEG-PDBA₆₀ and pH-dependent activation in vitro by a reporter cell assay.

FIG. 4B shows encapsulation of single chain human IL-12 with a protonated polymer intermediate of PEG-PDBA₁₇₀ and pH-dependent activation in vitro by a reporter cell assay.

FIG. 5A shows encapsulation of monovalent human IL-12Fc with a protonated polymer intermediate of PEG-PDBA₆₀ and pH-dependent activation in vitro by a reporter cell assay

FIG. 5B shows encapsulation of monovalent human IL-12Fc with a protonated polymer intermediate of PEG-PDBA₁₇₀ and pH-dependent activation in vitro by a reporter cell assay

FIG. 6A shows encapsulation of bivalent human IL-12Fc with a protonated polymer intermediate of PEG-PDBA₆₀ and pH-dependent activation in vitro by a reporter cell assay

FIG. 6B shows encapsulation of bivalent human IL-12Fc with a protonated polymer intermediate of PEG-PDBA₁₇₀ and pH-dependent activation in vitro by a reporter cell assay

FIG. 7A shows encapsulation of single chain mouse IL-12 with a protonated polymer intermediate of PEG-PDBA₆₀ and pH-dependent activation in vitro by a reporter cell assay.

FIG. 7B shows encapsulation of single chain mouse IL-12 with a protonated polymer intermediate of PEG-PDBA₉₀ and pH-dependent activation in vitro by a reporter cell assay.

FIG. 7C shows encapsulation of single chain mouse IL-12 with a protonated polymer intermediate of PEG-PDBA₁₂₀ and pH-dependent activation in vitro by a reporter cell assay.

FIG. 7D shows encapsulation of single chain mouse IL-12 with a protonated polymer intermediate of PEG-PDBA₁₇₀ and pH-dependent activation in vitro by a reporter cell assay.

FIG. 7E shows encapsulation of single chain mouse IL-12 with a protonated polymer intermediate of PEG-PDBA₂₀₀ and pH-dependent activation in vitro by a reporter cell assay.

FIG. 8A shows encapsulation of monovalent mouse IL-12Fc with a protonated polymer intermediate of PEG-PDBA₆₀ and pH-dependent activation in vitro by a reporter cell assay.

FIG. 8B shows encapsulation of monovalent mouse IL-12Fc with a protonated polymer intermediate of PEG-PDBA₉₀ and pH-dependent activation in vitro by a reporter cell assay.

FIG. 8C shows encapsulation of monovalent mouse IL-12Fc with a protonated polymer intermediate of PEG-PDBA₁₂₀ and pH-dependent activation in vitro by a reporter cell assay.

FIG. 8D shows encapsulation of monovalent mouse IL-12Fc with a protonated polymer intermediate of PEG-PDBA₁₇₀ and pH-dependent activation in vitro by a reporter cell assay.

FIG. 9A shows encapsulation of bivalent mouse IL-12Fc with a protonated polymer intermediate of PEG-PDBA₆₀ and pH-dependent activation in vitro by a reporter cell assay.

FIG. 9B shows encapsulation of bivalent mouse IL-12Fc with a protonated polymer intermediate of PEG-PDBA₉₀ and pH-dependent activation in vitro by a reporter cell assay.

FIG. 9C shows encapsulation of bivalent mouse IL-12Fc with a protonated polymer intermediate of PEG-PDBA₁₂₀ and pH-dependent activation in vitro by a reporter cell assay.

FIG. 9D shows encapsulation of bivalent mouse IL-12Fc with a protonated polymer intermediate of PEG-PDBA₁₄₀ and pH-dependent activation in vitro by a reporter cell assay.

FIG. 9E shows encapsulation of bivalent mouse IL-12Fc with a protonated polymer intermediate of PEG-PDBA₁₇₀ and pH-dependent activation in vitro by a reporter cell assay.

FIG. 10 is a table characterizing the IL-12 encapsulant formulations.

FIG. 11 shows encapsulation of bivalent human IL-2Fc with a protonated polymer intermediate of PEG-PDBA and pH-dependent activation in vitro by a reporter cell assay.

FIG. 12 shows encapsulation of human IL-18 with a protonated polymer intermediate of PEG-PDBA and pH-dependent activation in vitro by a reporter cell assay.

FIG. 13A shows encapsulation of solitomab (EPCAMxCD3 bispecific antibody) with a protonated polymer intermediate of PEG-PDBA and pH-dependent activation in vitro by a T cell dependent cellular cytotoxicity assay with SK-CO-1 cell line.

FIG. 13B shows encapsulation of solitomab (EPCAMxCD3 bispecific antibody) with a protonated polymer intermediate of PEG-PDBA and pH-dependent activation in vitro by a T cell dependent cellular cytotoxicity assay with GSU cell line.

FIG. 14A shows encapsulation of runimotamab (HER2xCD3 bispecific antibody) with a protonated polymer intermediate of PEG-PDBA and pH-dependent activation in vitro by a T cell dependent cellular cytotoxicity assay with GSU cell line.

FIG. 14B shows encapsulation of runimotamab (HER2xCD3 bispecific antibody) with a protonated polymer intermediate of PEG-PDBA and pH-dependent activation in vitro by a T cell dependent cellular cytotoxicity assay with HCC827 cell line.

FIG. 14C shows encapsulation of runimotamab (HER2xCD3 bispecific antibody) with a protonated polymer intermediate of PEG-PDBA and pH-dependent activation in vitro by a T cell dependent cellular cytotoxicity assay with SK-CO-1 cell line.

FIG. 15A shows encapsulation of blinatumomab (CD19xCD3 bispecific antibody) with a protonated polymer intermediate of PEG-PDBA and pH-dependent activation in vitro by a B cell depletion assay.

FIG. 15B shows encapsulation of odronextamab (CD20xCD3 bispecific antibody) with a protonated polymer intermediate of PEG-PDBA and pH-dependent activation in vitro by a B cell depletion assay.

FIG. 15C shows encapsulation of glofitamab (CD20xCD3 bispecific antibody) with a protonated polymer intermediate of PEG-PDBA and pH-dependent activation in vitro by a B cell depletion assay.

FIG. 16 is a table characterizing the bispecific encapsulant formulations.

FIG. 17 shows systemic cytokine levels in healthy BL6 mice on Day 5 after being intravenously administered two doses of PBS, free IL-12Fc proteins, or PEG-PDBA pH sensitive micelle encapsulated IL-12Fc (PDBA-IL-12Fc) on day 0 and day 3 of a study.

FIG. 18 shows levels of aspartate transaminase (AST), alanine transaminase (ALT), blood urea nitrogen (BUN), and creatinine (Cre) in healthy BL6 mice on Day 5 after being intravenously administered two doses of PBS, free IL-12Fc proteins, or PEG-PDBA pH sensitive micelle encapsulated IL-12Fc (PDBA-IL-12Fc) on day 0 and day 3 of a study.

FIG. 19 shows body weight change in healthy BL6 mice after being intravenously administered two doses of PBS, free IL-12Fc proteins, or PEG-PDBA pH sensitive micelle encapsulated IL-12Fc (PDBA-IL-12Fc) on day 0 and day 3 of a study.

FIG. 20A shows tumor volume measurements in mice bearing large tumors (˜500 mm³) after being intravenously administered a single dose of PBS, free IL-12Fc proteins, or PEG-PDBA pH sensitive micelle encapsulated IL-12Fc (PDBA-IL-12Fc) on day 0 of a study.

FIG. 20B shows body weight change in mice bearing large tumors (˜500 mm³) after being intravenously administered a single dose of PBS, free IL-12Fc proteins, or PEG-PDBA pH sensitive micelle encapsulated IL-12Fc (PDBA-IL-12Fc) on day 0 of a study.

FIG. 21 shows tumor volume and body weight loss measurements on day 7 of a study in mice bearing large tumors (˜500 mm³) after being intravenously administered a single dose of PBS, free IL-12Fc proteins, or PEG-PDBA pH sensitive micelle encapsulated IL-12Fc (PDBA-IL-12Fc) on day 0 of the study.

FIGS. 22A and 22B shows an increase in CD8 positive T cells and NK cells in the tumor of mice on day 2 of a study after being intravenously administered a single dose of PBS, free IL-12Fc proteins, or PEG-PDBA pH sensitive micelle encapsulated IL-12Fc (PDBA-IL-12Fc) on day 0 of the study.

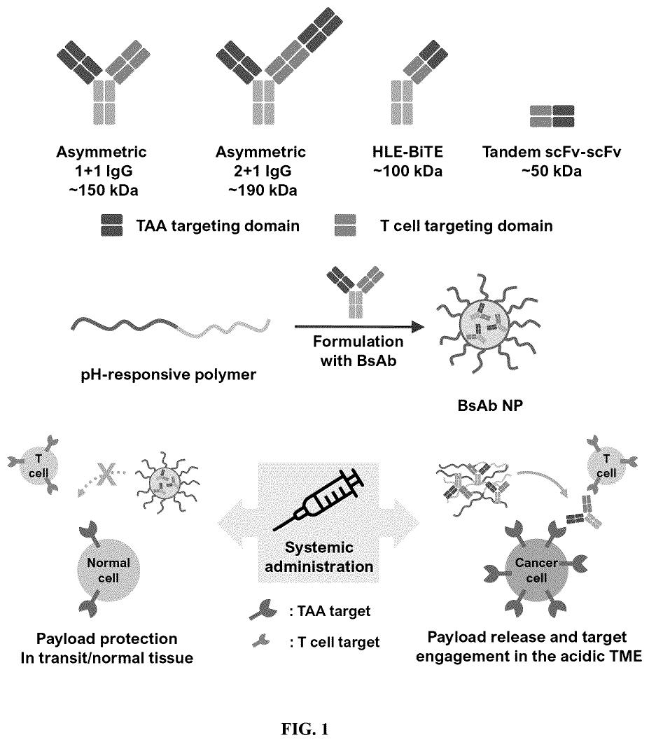

### DETAILED DESCRIPTION OF THE INVENTION

Provided herein are micelle compositions comprising a therapeutic agent. In some embodiments, the micelle comprises a diblock copolymer and a therapeutic agent. In other embodiments provided here in are micelle composition comprising a therapeutic agent.

### Definitions

In the following description, certain specific details are set forth in order to provide a thorough understanding of various embodiments. However, one skilled in the art will understand that the invention may be practiced without these details. In other instances, well-known structures have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed invention.

As used in this specification and the appended claims, the singular forms “a,”, an, and “the” include plural referents unless the content clearly dictates otherwise. It should also be noted that the term “or” is generally employed in its sense including “and/or” unless the content clearly dictates otherwise.

The terms below, as used herein, have the following meanings, unless indicated otherwise:

“Alkyl” refers to a straight or branched hydrocarbon chain radical, having from one to twenty carbon atoms, and which is attached to the rest of the molecule by a single bond. An alkyl comprising up to 10 carbon atoms is referred to as a C₁-C₁₀ alkyl, likewise, for example, an alkyl comprising up to 6 carbon atoms is a C₁-C₆ alkyl. Alkyls (and other moieties defined herein) comprising other numbers of carbon atoms are represented similarly. Alkyl groups include, but are not limited to, C₁-C₁₀ alkyl, C₁-C₉ alkyl, C₁-C₈ alkyl, C₁-C₇ alkyl, C₁-C₆ alkyl, C₁-C₅ alkyl, C₁-C₄ alkyl, C₁-C₃ alkyl, C₁-C₂ alkyl, C₂-C₈ alkyl, C₃-C₈ alkyl and C₄-C₈ alkyl. Representative alkyl groups include, but are not limited to, methyl, ethyl, n-propyl, 1-methylethyl (i-propyl), n-butyl, i-butyl, s-butyl, n-pentyl, 1,1-dimethylethyl (t-butyl), 3-methylhexyl, 2-methylhexyl, 1-ethyl-propyl, and the like. In some embodiments, the alkyl is methyl, ethyl, s-butyl, or 1-ethyl-propyl. Unless stated otherwise specifically in the specification, an alkyl group may be optionally substituted as described below. “Alkylene” or “alkylene chain” refers to a straight or branched divalent hydrocarbon chain linking the rest of the molecule to a radical group. In some embodiments, the alkylene is —CH₂—, —CH₂CH₂—, or —CH₂CH₂CH₂—. In some embodiments, the alkylene is —CH₂—. In some embodiments, the alkylene is —CH₂CH₂—. In some embodiments, the alkylene is —CH₂CH₂CH₂—.

“Aryl” refers to an aromatic ring wherein each of the atoms forming the ring is a carbon atom. Aryl groups can be optionally substituted. Examples of aryl groups include, but are not limited to phenyl, and naphthalenyl. In some embodiments, the aryl is phenyl. Depending on the structure, an aryl group can be a monoradical or a diradical (i.e., an arylene group). Unless stated otherwise specifically in the specification, the term “aryl” or the prefix “ar-” (such as in “aralkyl”) is meant to include aryl radicals that are optionally substituted.

“Cycloalkyl” refers to a monocyclic or polycyclic non-aromatic radical, wherein each of the atoms forming the ring (i.e. skeletal atoms) is a carbon atom. Cycloalkyls may be saturated, or partially unsaturated. Cycloalkyls may be fused with an aromatic ring (in which case the cycloalkyl is bonded through a non-aromatic ring carbon atom). Cycloalkyl groups include groups having from 3 to 10 ring atoms. In some embodiments, a cycloalkyl is a C₃-C₆ cycloalkyl. In some embodiments, a cycloalkyl is a 3- to 6-membered cycloalkyl. Representative cycloalkyls include, but are not limited to, cycloakyls having from three to ten carbon atoms, from three to eight carbon atoms, from three to six carbon atoms, or from three to five carbon atoms. Monocyclic cyclcoalkyl radicals include, for example, cyclopropyl, cyclobutyl, cyclopentyl, cyclohexyl, cycloheptyl, and cyclooctyl. In some embodiments, the monocyclic cyclcoalkyl is cyclopropyl, cyclobutyl, cyclopentyl or cyclohexyl. Polycyclic radicals include, for example, adamantyl, norbornyl, decalinyl, and 3,4-dihydronaphthalen-1(2H)-one. Unless otherwise stated specifically in the specification, a cycloalkyl group may be optionally substituted.

The term “optionally substituted” or “substituted” means that the referenced group may be substituted with one or more additional group(s) individually and independently selected from alkyl, haloalkyl, cycloalkyl, aryl, heteroaryl, heterocycloalkyl, —OH, alkoxy, aryloxy, alkylthio, arylthio, alkylsulfoxide, arylsulfoxide, alkylsulfone, arylsulfone, —CN, alkyne, C₁-C₆alkylalkyne, halogen, acyl, acyloxy, —CO₂H, —CO₂alkyl, nitro, and amino, including mono- and di-substituted amino groups (e.g., —NH₂, —NHR, —N(R)₂), and the protected derivatives thereof. In some embodiments, optional substituents are independently selected from alkyl, alkoxy, haloalkyl, cycloalkyl, halogen, —CN, —NH₂, —NH(CH₃), —N(CH₃)₂, —OH, —CO₂H, and —CO₂alkyl. In some embodiments, optional substituents are independently selected from fluoro, chloro, bromo, iodo, —CH₃, —CH₂CH₃, —CF₃, —OCH₃, and —OCF₃. In some embodiments, optional substituents are independently selected from fluoro, chloro, —CH₃, —CF₃, —OCH₃, and —OCF₃. In some embodiments, substituted groups are substituted with one or two of the preceding groups. In some embodiments, an optional substituent on an aliphatic carbon atom (acyclic or cyclic, saturated or unsaturated carbon atoms, excluding aromatic carbon atoms) includes oxo (═O).

The terms “co-administration” or the like, as used herein, are meant to encompass administration of the selected therapeutic agents to a single patient, and are intended to include treatment regimens in which the agents are administered by the same or different route of administration or at the same or different time.

The terms “effective amount” or “therapeutically effective amount,” as used herein, refer to a sufficient amount of an agent or a compound being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case may be determined using techniques, such as a dose escalation study.

Unless otherwise stated, the following terms used in this application have the definitions given below. The use of the term “including” as well as other forms, such as “include”, “includes,” and “included,” is not limiting. The section headings used herein are for organizational purposes only and are not to be construed as limiting the subject matter described.

“Pharmaceutically acceptable,” as used herein, refers a material, such as a carrier or diluent, which does not abrogate the biological activity or properties of the block copolymer, and is relatively nontoxic, i.e., the material is administered to an individual without causing undesirable biological effects or interacting in a deleterious manner with any of the components of the composition in which it is contained.

The term “pharmaceutically acceptable salt” refers to a form of a therapeutically active agent that consists of a cationic form of the therapeutically active agent in combination with a suitable anion, or in alternative embodiments, an anionic form of the therapeutically active agent in combination with a suitable cation. Handbook of Pharmaceutical Salts: Properties, Selection and Use. International Union of Pure and Applied Chemistry, Wiley-VCH 2002. S. M. Berge, L. D. Bighley, D. C. Monkhouse, J. Pharm. Sci. 1977, 66, 1-19. P. H. Stahl and C. G. Wermuth, editors, Handbook of Pharmaceutical Salts: Properties, Selection and Use, Weinheim/Zurich:Wiley-VCH/VHCA, 2002. Pharmaceutical salts typically are more soluble and more rapidly soluble in stomach and intestinal juices than non-ionic species and so are useful in solid dosage forms. Furthermore, because their solubility often is a function of pH, selective dissolution in one or another part of the digestive tract is possible and this capability can be manipulated as one aspect of delayed and sustained release behaviors. Also, because the salt-forming molecule can be in equilibrium with a neutral form, passage through biological membranes can be adjusted.

As used herein, “pH responsive system,” “pH responsive composition,” “micelle,” “pH-responsive micelle,” “pH-sensitive micelle,” “pH-activatable micelle”, “pH-sensitive encapsulant”, “pH-activatable encapsulant”, “pH-responsive encapsulant”, and “pH-activatable micellar (pHAM) nanoparticle” are used interchangeably herein to indicate a micelle comprising one or more compounds, which disassociates depending on the pH (e.g., above or below a certain pH). As a non-limiting example, at a certain pH, the block copolymers of Formula (I) is substantially in micellar form. As the pH changes (e.g., decreases), the micelles begin to disassociate, and as the pH further changes (e.g., further decreases), the block copolymers of Formula (I) is present substantially in disassociated (non-micellar) form.

As used herein, “encapsulation” or “encapsulation process” are used interchangeably herein to indicate the formation of a micelle.

As used herein, “pH transition range” indicates the pH range over which the micelles disassociate.

As used herein, “pH transition value” (pH) indicates the pH at which half of the micelles are disassociated.

A “nanoprobe” is used herein to indicate a pH-sensitive micelle which comprises an imaging labeling moiety. In some embodiments, the labeling moiety is a fluorescent dye. In some embodiments, the fluorescent dye is indocyanine green dye.

The terms “administer,” “administering”, “administration,” and the like, as used herein, refer to the methods that may be used to enable delivery of compounds or compositions to the desired site of biological action. These methods include, but are not limited to oral routes, intraduodenal routes, parenteral injection (including intravenous, subcutaneous, intraperitoneal, intramuscular, intravascular or infusion), topical and rectal administration. Those of skill in the art are familiar with administration techniques that can be employed with the compounds and methods described herein. In some embodiments, the compounds and compositions described herein are administered orally. In some embodiments, the compositions described herein are administered intravenously.

The terms “co-administration” or the like, as used herein, are meant to encompass administration of the selected therapeutic agents to a single patient, and are intended to include treatment regimens in which the agents are administered by the same or different route of administration or at the same or different time.

The terms “effective amount” or “therapeutically effective amount,” as used herein, refer to a sufficient amount of an agent or a compound being administered, which will relieve to some extent one or more of the symptoms of the disease or condition being treated. The result includes reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case is optionally determined using techniques, such as a dose escalation study.

The terms “enhance” or “enhancing,” as used herein, means to increase or prolong either in potency or duration a desired effect. Thus, in regard to enhancing the effect of therapeutic agents, the term “enhancing” refers to the ability to increase or prolong, either in potency or duration, the effect of other therapeutic agents on a system. An “enhancing-effective amount,” as used herein, refers to an amount adequate to enhance the effect of another therapeutic agent in a desired system.

The term “subject” or “patient” encompasses mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human.

The terms “treat,” “treating” or “treatment,” as used herein, include alleviating, abating or ameliorating at least one symptom of a disease or condition, preventing additional symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the standard deviation of error for the device or method being employed to determine the value. Following longstanding patent law, the words “a” and “an,” when used in conjunction with the word “comprising” in the claims or specification, denotes one or more, unless specifically noted.

I. Micelles

One or more block copolymers described herein may be used to form a pH-sensitive micelle or encapsulant. In some embodiments, a composition comprises a single type of micelle. In some embodiments, two or more different types of micelles may be combined to form a mixed-micelle composition. In some embodiments, the micelle comprises one or more block copolymer that non-covalently encapsulates a therapeutic agent.

In certain embodiments provided herein is a micelle, comprising:

(i) Block Copolymers

In some embodiments, the block copolymer of Formula (I) comprises poly(ethylene oxide) (PEO), and a hydrophobic polymer segment with the following structure:

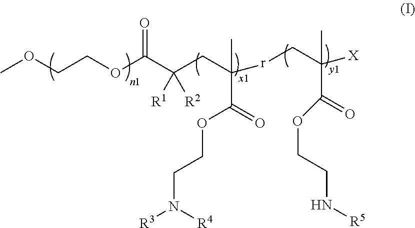

wherein: n₁ is an integer from 40-500, x₁ is an integer from 4-250, y₁ is an integer from 0-10, X is a halogen, —OH, or —C(O)OH, R¹ and R² are each independently hydrogen or optionally substituted C₁-C₆ alkyl, R³ and R⁴ are each independently an optionally substituted C₁-C₆ alkyl, C₃-C₁₀ cycloalkyl or aryl, or R³ and R⁴ are taken together with the corresponding nitrogen to which they are attached form an optionally substituted 5 to 7-membered ring, R⁵ is hydrogen or —C(O)CH₃. 5. In some embodiments, n1 is an integer from 100-250, x1 is an integer from 40-250, and/or y1 is 0. In some embodiments, n1 is an integer from 100-250, x1 is an integer from 100-200, and/or y1 is 0. In some embodiments, n1 is an integer of about 114, x1 is about 170, and/or y1 is 0. In some embodiments, n1 is an integer of 114, x1 is 170, and/or y1 is 0. The units of x1 may include the same units having the same R³ and R⁴ substituents or different units which have different R³ and R⁴ substituents. Further, the addition of other hydrophobic monomeric units in small quantities that do not significantly affect the ability of the micelle to encapsulate and release a biomolecule composition should be understood to be included in Formula (I).

In some embodiments, the hydrophobic polymer segment of the block copolymer of formula (I) is selected from:

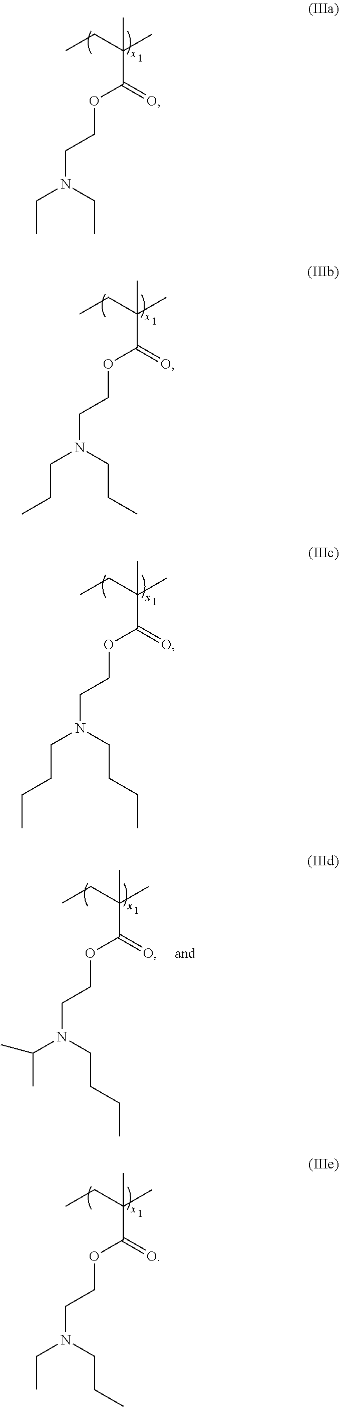

(ii) Therapeutic Agents

Bispecific antibodies (BsAbs) are an important class of therapeutics for immune-oncology applications. T cell engagers (TCEs) target tumor associated antigens and T cells to eradicate antigen-expressing tumor cells. TCEs for solid tumors have likewise demonstrated encouraging clinical efficacy but shown dose-limiting toxicities due to on-target/off-tumor effects.

Cytokines (e.g., IL-12, IL-2) and cytokine fusion proteins (e.g., IL-12Fc, IL-2Fc) can induce anti-tumor immune responses, but their clinical applications are limited by the unfavorable pharmacokinetic properties and serious dose-limiting toxicities (e.g., cytokine release syndrome, vascular leak syndrome, etc).

In some embodiments, the therapeutic agent is a biomolecule. In some embodiments, the therapeutic agent is a protein. In some embodiments, the therapeutic agent is a bispecific antibody (BsAbs). In some embodiments, the therapeutic agent is a bispecific antibody with a TAA targeting domain and a T cell targeting domain. In some embodiments the therapeutic agent is a cytokine. In some embodiments, the therapeutic agent is a asymmetric 1+1 IgG bispecific antibody. In some embodiments, the therapeutic agent is agent is asymmetric 2+1 IgG bispecific antibody. In some embodiments, the therapeutic agent is HLE-BiTE bispecific antibody. In some embodiments, the therapeutic agent is agent is tandem scFv-scFv bispecific antibody. In some embodiments, the therapeutic agent is a protein having a molecular weight of at least 6 kDa.

In some embodiments the therapeutic agent is a human IL-12. In some embodiments the therapeutic agent is single chain human IL-12. In some embodiments the therapeutic agent is monovalent human IL-12 fused to the Fc region of the IgG antibody. In some embodiments the therapeutic agent is bivalent human IL-12 fused to the Fc region of the IgG antibody. In some embodiments the therapeutic agent is human IL-2. In some embodiments the therapeutic agent is bivalent human IL-2 fused to the Fc region of the IgG antibody. In some embodiments the therapeutic agent is a human IL-18. In some embodiments the therapeutic agent is a solitomab bispecific antibody T cell engager (TCE). In some embodiments the therapeutic agent is a runimotamab bispecific antibody T cell engager. In some embodiments the therapeutic agent is a blinatumomab bispecific antibody T cell engager. In some embodiments the therapeutic agent is glofitamab bispecific antibody T cell engager. In some embodiments the therapeutic agent is a odronextamab bispecific antibody T cell engager.

Monovalent human IL12(p35/p40)-hIgG1-
Fc-LALA/PG heterodimer sequences
Human IL12-p35-hIgG1-Fc-knob
(L234A/L235A/P329G/S354C/T366W)
I.
SEQ ID NO: 1
RNLPVATPDPGMFPCLHHSQNLLRAVSNMLQKARQTLEFY
PCTSEEIDHEDITKDKTSTVEACLPLELTKNESCLNSRET
SFITNGSCLASRKTSFMMALCLSSIYEDLKMYQVEFKTMN
AKLLMDPKRQIFLDQNMLAVIDELMQALNFNSETVPQKSS
LEEPDFYKTKIKLCILLHAFRIRAVTIDRVMSYLNASGGG
GSGGGGSPKSCDKTHTCPPCPAPEAAGGPSVFLFPPKPKD
TLMISRTPEVTCVVVDVSHEDPEVKFNWYVDGVEVHNAKT
KPREEQYNSTYRVVSVLTVLHQDWLNGKEYKCKVSNKALG
APIEKTISKAKGQPREPQVYTLPPCRDELTKNQVSLWCLV
KGFYPSDIAVEWESNGQPENNYKTTPPVLDSDGSFFLYSK
LTVDKSRWQQGNVFSCSVMHEALHNHYTQKSLSLSPGK
Human IL12-p40-hIgG1-Fc-hole
(L234A/L235A/P329G/Y349C/T366S/L368A/Y407V)
I.
SEQ ID NO: 2
IWELKKDVYVVELDWYPDAPGEMVVLTCDTPEEDGITWTL
DQSSEVLGSGKTLTIQVKEFGDAGQYTCHKGGEVLSHSLL
LLHKKEDGIWSTDILKDQKEPKNKTFLRCEAKNYSGRFTC
WWLTTISTDLTFSVKSSRGSSDPQGVTCGAATLSAERVRG
DNKEYEYSVECQEDSACPAAEESLPIEVMVDAVHKLKYEN
YTSSFFIRDIIKPDPPKNLQLKPLKNSRQVEVSWEYPDTW
STPHSYFSLTFCVQVQGKSKREKKDRVFTDKTSATVICRK
NASISVRAQDRYYSSSWSEWASVPCSGGGGSGGGGSPKSC
DKTHTCPPCPAPEAAGGPSVFLFPPKPKDTLMISRTPEVT
CVVVDVSHEDPEVKFNWYVDGVEVHNAKTKPREEQYNSTY
RVVSVLTVLHQDWLNGKEYKCKVSNKALGAPIEKTISKAK
GQPREPQVCTLPPSRDELTKNQVSLSCAVKGFYPSDIAVE
WESNGQPENNYKTTPPVLDSDGSFFLVSKLTVDKSRWQQG
NVFSCSVMHEALHNHYTQKSLSLSPGK
Bivalent human IL12(p40/p35)-hIgG1-Fc-
LALA/PG homodimer sequence
Human IL12-p40-G4S linker-P35-hIgG1-Fc
(L234A/L235A/P329G)
I.
SEQ ID NO: 3
IWELKKDVYVVELDWYPDAPGEMVVLTCDTPEEDGITWTL
DQSSEVLGSGKTLTIQVKEFGDAGQYTCHKGGEVLSHSLL
LLHKKEDGIWSTDILKDQKEPKNKTFLRCEAKNYSGRFTC
WWLTTISTDLTFSVKSSRGSSDPQGVTCGAATLSAERVRG
DNKEYEYSVECQEDSACPAAEESLPIEVMVDAVHKLKYEN
YTSSFFIRDIIKPDPPKNLQLKPLKNSRQVEVSWEYPDTW
STPHSYFSLTFCVQVQGKSKREKKDRVFTDKTSATVICRK
NASISVRAQDRYYSSSWSEWASVPCSGGGGSGGGGSRNLP
VATPDPGMFPCLHHSQNLLRAVSNMLQKARQTLEFYPCTS
EEIDHEDITKDKTSTVEACLPLELTKNESCLNSRETSFIT
NGSCLASRKTSFMMALCLSSIYEDLKMYQVEFKTMNAKLL
MDPKRQIFLDQNMLAVIDELMQALNFNSETVPQKSSLEEP
DFYKTKIKLCILLHAFRIRAVTIDRVMSYLNASGGGGSGG
GGSPKSCDKTHTCPPCPAPEAAGGPSVFLFPPKPKDTLMI
SRTPEVTCVVVDVSHEDPEVKFNWYVDGVEVHNAKTKPRE
EQYNSTYRVVSVLTVLHQDWLNGKEYKCKVSNKALGAPIE
KTISKAKGQPREPQVYTLPPSRDELTKNQVSLTCLVKGFY
PSDIAVEWESNGQPENNYKTTPPVLDSDGSFFLYSKLTVD
KSRWQQGNVFSCSVMHEALHNHYTQKSLSLSPGK
Single chain human IL12 heterodimer sequence
Human IL12-p40-(G4S)5-P35-HIS tag
I.
SEQ ID NO: 4
IWELKKDVYVVELDWYPDAPGEMVVLTCDTPEEDGITWTL
DQSSEVLGSGKTLTIQVKEFGDAGQYTCHKGGEVLSHSLL
LLHKKEDGIWSTDILKDQKEPKNKTFLRCEAKNYSGRFTC
WWLTTISTDLTFSVKSSRGSSDPQGVTCGAATLSAERVRG
DNKEYEYSVECQEDSACPAAEESLPIEVMVDAVHKLKYEN
YTSSFFIRDIIKPDPPKNLQLKPLKNSRQVEVSWEYPDTW
STPHSYFSLTFCVQVQGKSKREKKDRVFTDKTSATVICRK
NASISVRAQDRYYSSSWSEWASVPCSGGGGSGGGGSGGGG
SGGGGSGGGGSRNLPVATPDPGMFPCLHHSQNLLRAVSNM
LQKARQTLEFYPCTSEEIDHEDITKDKTSTVEACLPLELT
KNESCLNSRETSFITNGSCLASRKTSFMMALCLSSIYEDL
KMYQVEFKTMNAKLLMDPKRQIFLDQNMLAVIDELMQALN
FNSETVPQKSSLEEPDFYKTKIKLCILLHAFRIRAVTIDR
VMSYLNASHHHHHH
Wild type human IL-12 sequences
IL-12 p35 (Arg23-Ser219) Accession # P29459
I.
SEQ ID NO: 5
RNLPVATPDPGMFPCLHHSQNLLRAVSNMLQKARQTLEFY
PCTSEEIDHEDITKDKTSTVEACLPLELTKNESCLNSRET
SFITNGSCLASRKTSFMMALCLSSIYEDLKMYQVEFKTMN
AKLLMDPKRQIFLDQNMLAVIDELMQALNFNSETVPQKSS
LEEPDFYKTKIKLCILLHAFRIRAVTIDR VMSYLNAS
IL-12 p40 (Ile23-Ser328) Accession # P29460
I.
SEQ ID NO: 6
IWELKKDVYVVELDWYPDAPGEMVVLTCDTPEEDGITWTL
DQSSEVLGSGKTLTIQVKEFGDAGQYTCHKGGEVLSHSLL
LLHKKEDGIWSTDILKDQKEPKNKTFLRCEAKNYSGRFTC
WWLTTISTDLTFSVKSSRGSSDPQGVTCGAATLSAERVRG
DNKEYEYSVECQEDSACPAAEESLPIEVMVDAVHKLKYEN
YTSSFFIRDIIKPDPPKNLQLKPLKNSRQVEVSWEYPDTW
STPHSYFSLTFCVQVQGKSKREKKDRVFTDKTSATVICRK
NASISVRAQDRYYSSSWSEWASVPCS
IL-2Fc Homodimer sequence
IL2-(G4S)1-Fc
I.
SEQ ID NO: 7
APTSSSTKKTQLQLEHLLLDLQMILNGINNYKNPKLTRML
TFKFYMPKKATELKHLQCLEEELKPLEEVLNLAQSKNFHL
RPRDLISNINVIVLELKGSETTFMCEYADETATIVEFLNR
WITFCQSIISTLTGGGGSEPKSSDKTHTCPPCPAPELLGG
PSVFLFPPKPKDTLMISRTPEVTCVVVDVSHEDPEVKFNW
YVDGVEVHNAKTKPREEQYNSTYRVVSVLTVLHQDWLNGK
EYKCKVSNKALPAPIEKTISKAKGQPREPQVYTLPPSRDE
LTKNQVSLTCLVKGFYPSDIAVEWESNGQPENNYKTTPPV
LDSDGSFFLYSKLTVDKSRWQQGNVFSCSVMHEALHNHYT
QKSLSLSPGK
Wild type human IL-18 sequence
NCBI Accession nNP_001553.1
(Predicted N-term AA Y37)
I.
SEQ ID NO: 8
MAAEPVEDNCINFVAMKFIDNTLYFIAEDDENLESDYFGK
LESKLSVIRNLNDQVLFIDQGNRPLFEDMTDSDCRDNAPR
TIFIISMYKDSQPRGMAVTISVKCEKISTLSCENKIISFK
EMNPPDNIKDTKSDIIFFQRSVPGHDNKMQFESSSYEGYF
LACEKERDLFKLILKKEDELGDRSIMFTVQNED

II. Method of Encapsulation

To formulate drug-loaded micelles, the polymer is dissolved in an organic solvent and the polymer is protonated with an acid. After protonation, the organic solvent and excess acid are removed. The therapeutic agent is dispensed in an aqueous buffer and mixed with the protonated polymer. The mixture is then dialyzed against a neutral buffer to complete the encapsulation process.

Encapsulation of a therapeutic payload is achieved using an acid protonated polymer intermediate. The protonation of the polymer generates a strong positive charge on a region of the polymer. The positively charged region of the polymer attracts a negatively charged region in the therapeutic payload. The electrostatic interaction between the positively charged polymer and negatively charged therapeutic payload creates a physical approximation between the polymer chains and the biomolecule. Neutralization of the polymer and therapeutic payload results in a sudden increase of hydrophobicity of the positively charged polymer section which interacts with the hydrophobic regions in the therapeutic payload to form a stable encapsulated structure or micelle that exhibits an in vitro pH-dependent activation window.

In an embodiment, the steps of encapsulating a therapeutic agent comprises of:
(i) dissolving a polymer in an organic solvent
(ii) protonating the polymer of (i) with an acid
(iii) removing excess organic solvent and acid of (ii)
(iv) adding the therapeutic agent to the polymer of (iii)
(v) dialyzing the mixture of (iv) against a neutral buffer.

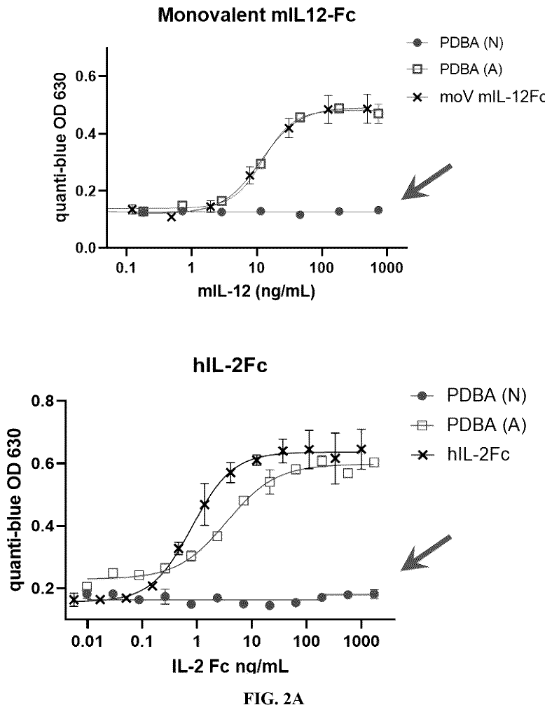

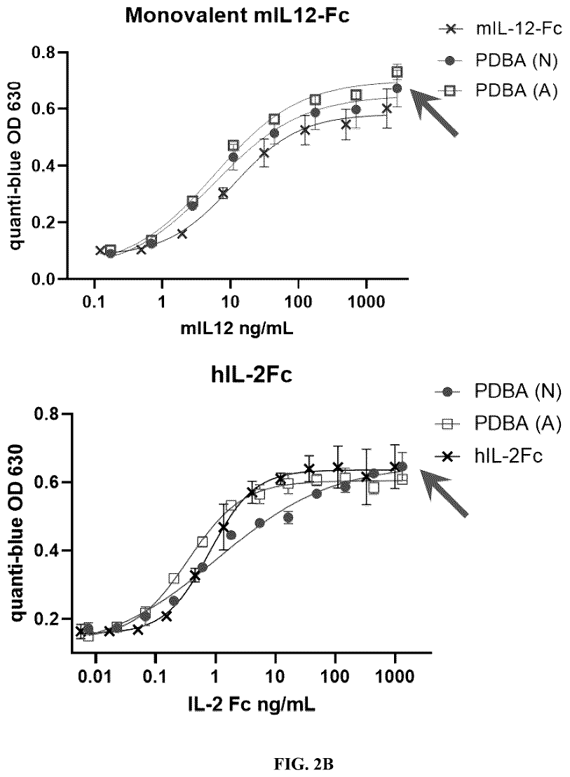

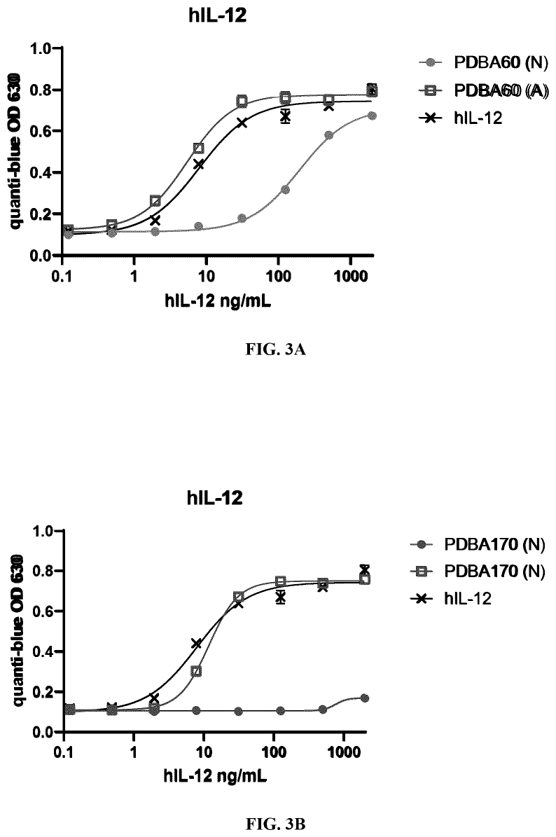

In an embodiment, the polymer is a PEG-PDBA polymer, and it is dissolved in methanol and protonated by acetic acid. An Amicon Ultra 10k MWCO device is used to remove the organic solvent and excess acetic acid. The therapeutic agent, a protein, is dispensed in 1×PBS or 10 mM sodium phosphate buffer and mixed and incubated overnight with gentle rocking. The mixture is then dialyzed against 10 mM sodium phosphate buffer at pH 7.4 to complete the encapsulation process. The step of protonating the polymer intermediate enables the characteristic of a large activation window as seen in FIG. 2A. The same method without the step of protonating the polymer results in a small activation or nonexistent activation window as seen in FIG. 2B.

In some embodiments, the therapeutic agent is about 0.1% wt of the micelle. In some embodiments the therapeutic agent is about 0.2%, about 0.3%, about 0.4%, about 0.5%, about 0.6%, about 0.7%, about 0.8%, about 0.9%, or about 1% of the micelle. In some embodiments the therapeutic agent is about 2%, about 3%, about 4%, about 5%, about 6%, about 7%, about 8%, about 9%, or about 10% of the micelle. In some embodiments the therapeutic agent is about 12%, about 14%, about 16%, about 18%, or about 20% of the micelle.

III. pH Responsive Compositions

In another aspect presented herein, are pH responsive compositions. The pH responsive compositions disclosed herein, comprise one or more pH-responsive micelles and/or nanoparticles that comprise block copolymers and a therapeutic agent. Each block copolymer comprises a hydrophilic polymer segment and a hydrophobic polymer segment wherein the hydrophobic polymer segment comprises an ionizable amine group to render pH sensitivity. This pH sensitivity is exploited to provide compositions suitable as drug-encapsulated therapeutics.

The micelles may have different pH transition values within physiological range, in order to target specific cells or microenvironments. In some embodiments, the micelle has a pH transition value of about 5 to about 8. In some embodiments, the micelle has a pH transition value of about 5 to about 6. In some embodiments, the micelle has a pH transition value of about 6 to about 7. In some embodiments, the micelle has a pH transition value of about 7 to about 8. In some embodiments, the micelle has a pH transition value of about 6.3 to about 6.9. In some embodiments, the micelle has a pH transition value of about 5.0 to about 6.2. In some embodiments, the micelle has a pH transition value of about 5.9 to about 6.2. In some embodiments, the micelle has a pH transition value of about 5.0 to about 5.5. In some embodiments, the pH transition point is 4.8, 4.9, 5.0, 5.1, 5.2, 5.3, 5.4, or 5.5.

The pH-sensitive micelle compositions of the invention may advantageously have a narrow pH transition range, in contrast to other pH sensitive compositions in which the pH response is very broad (i.e. 2 pH units). This pH transition is the transition point at which the micelle dissociates, releasing the payload or activating the photophore (i.e., an indocyanine green dye). In some embodiments, the micelles have a pH transition range of less than about 1 pH unit. In various embodiments, the micelles have a pH transition range of less than about 0.9, less than about 0.8, less than about 0.7, less than about 0.6, less than about 0.5, less than about 0.4, less than about 0.3, less than about 0.2, less than about 0.1 pH unit. In some embodiments, the micelles have a pH transition range of less than about 0.5 pH unit. In some embodiments, the micelles have a pH transition range of less than about 0.25 pH unit. The narrow pH transition range advantageously provides a sharper pH response that can result in complete turn-on of the fluorophores or release the therapeutic payload with subtle changes of pH.

In some embodiments, the pH responsive compositions have an emission spectrum. In some embodiments, the emission spectrum is from 600-800 nm. In some embodiments, the emission spectrum is from 700-800 nm.

IV. Methods of Use

Aerobic glycolysis, known as the Warburg effect, in which cancer cells preferentially uptake glucose and convert it into lactic acid or other acids, occurs in all solid cancers. Lactic acid or other acids preferentially accumulates in the extracellular space due to monocarboxylate transporters or other transporters. The resulting acidification of the extracellular space promotes remodeling of the extracellular matrix for further tumor invasion and metastasis.

Some embodiments provided herein describe compounds that form micelles at physiologic pH (7.35-7.45). In some embodiments, the compounds described herein are non-covalently incorporated to a therapeutic agent. In some embodiments, the therapeutic agents are sequestered within the micelle core at physiologic pH (7.35-7.45) (e.g., during blood circulation). In some embodiments, when the micelle encounters an acidic environment (e.g., tumor tissues), the micelles dissociate into individual compounds allowing the release of the therapeutic agent. In some embodiments, the micelle dissociates at a pH below the pH transition point (e.g., the acidic state of tumor microenvironment).

In some embodiments, the therapeutic agent may be incorporated into the interior of the micelles. Specific pH conditions (e.g. acidic pH present in tumors and endocytic compartments) may lead to rapid protonation and dissociation of micelles into unimers, thereby releasing the therapeutic agent (e.g. a drug). In some embodiments, the micelle provides stable drug encapsulation at physiological pH (pH 7.4), but can quickly release the drug in acidic environments.

In some instances, the pH-sensitive micelle compositions described herein have a narrow pH transition range. In some embodiments, the micelles described herein have a pH transition range (ΔpH₁₀₋₉₀%) of less than 1 pH unit. In various embodiments, the micelles have a pH transition range of less than about 0.9, less than about 0.8, less than about 0.7, less than about 0.6, less than about 0.5, less than about 0.4, less than about 0.3, less than about 0.2, less than about 0.1 pH unit. In some embodiments, the micelles have a pH transition range of less than about 0.5 pH unit. In some embodiments, the pH transition range is less than 0.25 pH units. In some embodiments, the pH transition range is less than 0.15 pH units. A sharp transition point allows the micelles to dissociate with the acidic tumor microenvironment.

These micelles may be used as drug-delivery agents. Micelles comprising a drug may be used to treat e.g., cancers, or other diseases wherein the drug may be delivered to the appropriate location due to localized pH differences (e.g., a pH different from physiological pH (7.4)). In some embodiments, the disorder treated is cancer. In some embodiments, the cancer comprises a solid tumor. In some embodiments, the tumor is a secondary tumor from metastasis of a primary tumor(s). In some embodiments, the drug-delivery may be to a lymph node or to a pleural surface.

In some embodiments of the methods disclosed herein, the tumor is from a cancer. In some embodiments, the cancer is breast cancer, head and neck squamous cell carcinoma (NHSCC), lung cancer, ovarian cancer, prostate cancer, bladder cancer, urethral cancer, esophageal cancer, colorectal cancer, peritoneal metastasis, renal cancer, or brain, skin (including melanoma and sarcoma). In some embodiments, the cancer is breast cancer, head and neck squamous cell carcinoma (NHSCC), esophageal cancer, colorectal cancer, or renal cancer.

In some embodiments, the tumor is reduced by about 5%, about 10%, about 15%, about 25%, about 30%, about 40%, about 50%, about 60%, about 70%, about 80%, or about 90%. In some embodiments, the tumor is reduced by about 50%. In some embodiments, the tumor is reduced by about 60%. In some embodiments, the tumor is reduced by about 70%. In some embodiments, the tumor is reduced by about 75%. In some embodiments, the tumor is reduced by about 80%. In some embodiments, the tumor is reduced by about 85%. In some embodiments, the tumor is reduced by about 90%. In some embodiments, the tumor is reduced by about 95%. In some embodiments, the tumor is reduced by about 99%.

V. Combination Therapy

In another aspect, the micelles comprising a block copolymer of PEG-PDBA, or a pharmaceutically acceptable salt, solvate, or hydrate thereof; and a therapeutic agent further comprise the administration of one or more additional therapies. In some embodiments, the additional therapy is checkpoint inhibitor. Checkpoint inhibitor therapy is a form of cancer immunotherapy. The therapy targets immune checkpoints, key regulators of the immune system that when stimulated can dampen the immune response to an immunologic stimulus. Some cancers can protect themselves from attack by stimulating immune checkpoint targets. Checkpoint therapy can block inhibitory checkpoints, restoring immune system function. Examples of checkpoint proteins found on T-cells or cancer cells include PD-1/PD-L1 and CTLA-4/B7-1/B7-2. Some immune checkpoint inhibitors are used to treat cancer.

PD-1 inhibitors and PD-L1 inhibitors are a group of checkpoint inhibitor anticancer drugs that block the activity of PD-1 and PDL1 immune checkpoint proteins present on the surface of cells. Immune checkpoint inhibitors are emerging as a front-line treatment for several types of cancer.

In some embodiments, the additional therapy is a checkpoint inhibitor. In some embodiments, the checkpoint inhibitor is an anti-PD-1 therapy, anti-PD-L1 therapy, or anti-CTLA-4 therapy. In some embodiments, the checkpoint inhibitor is an anti-PD-1 therapy.

In some embodiments, the additional therapy is selected from Pembrolizumab (Keytruda), Nivolumab (Opdivo), Cemiplimab (Libtayo), and Durvalumab (Imfinzi); or any combination thereof. In some embodiments, the additional therapy is Pembrolizumab or Keytruda. In some embodiments, the additional therapy is Nivolumab or Opdivo. In some embodiments, the additional therapy is Durvalumab or Imfinzi. In some embodiments, the additional therapy is Cemiptimab or Libtayo.

The additional therapy may be administered concurrently or sequentially with the pH responsive composition described herein.

### EXAMPLES

Block copolymers and micelles described herein are synthesized using standard synthetic techniques or using methods known in the art.

Unless otherwise indicated, conventional methods of mass spectroscopy, NMR, HPLC, protein chemistry, biochemistry, recombinant DNA techniques and pharmacology are employed.

Block copolymers are prepared using standard organic chemistry techniques such as those described in, for example, March's Advanced Organic Chemistry, 6^{th} Edition, John Wiley and Sons, Inc.

Some abbreviations used herein are as follows:

| | | |
|---|---|---|
| I. | MeOH: | methanol |
| II. | PEG | Polyethylene glycol |
| III. | PEO | Polyethylene oxide |
| IV. | PDBA | Poly(2-(dibutylamino)ethyl methacrylate) |
| V. | Hr | Hour(s) |
| VI. | ISR | Incurred sample reanalysis |
| VII. | kg | Kilogram |
| VIII. | mg | Milligram(s) |
| IX. | mL | Milliliters(s) |
| X. | NP | Nanoparticle |
| XI. | μg | Microgram(s) |
| XII. | μm | Mircon(s) |
| XIII. | UPS | Ultra pH-sensitive |
| XIV. | BsAbs | Bispecific Antibodies |
| XV. | TCE | T-cell engager |
| XVI. | IL | Interleukin |
| XVII. | PDI | Polydispersity index |
| XVIII. | WFI | Water-for-injection |

Suitable PEG polymers may be purchased (for example, from Sigma Aldrich) or may be synthesized according to methods known in the art. In some embodiments, the hydrophilic polymer can be used as an initiator for polymerization of the hydrophobic monomers to form a block copolymer.

### Example 1. Encapsulation of a Single Chain Mouse IL-12 Using PEG-PDBA Polymer Via a Protonated Polymer Intermediate

To formulate drug-loaded micelles, the PEG-PDBA polymer was first dispersed into methanol at a concentration of 10 mg/mL. 1.05 equivalents of acetic acid per ionizable amine moiety on the polymer was added to protonate and dissolve the polymer. An Amicon Ultra 10k MWCO device was used to remove organic solvent and excess acid from the polymer after protonation by diluting the polymer solution in WFI to the capacity of the Amicon Ultra device, centrifuging, and discarding the permeate. This process was repeated 7 times. The polymer was concentrated to 10 mg/mL at the end of the process. The single chain mouse IL-12 at a concentration of 14.22 mg/mL with the desired quantity (0.1%-20% of polymer weight) was then added to the protonated polymer intermediate from above, mixed gently and incubated overnight with gentle rocking. The mixture was then dialyzed against a neutral buffer, e.g. 10 mM sodium phosphate buffer, at pH 7.4 to complete the encapsulation process.

### Example 2. Encapsulation of a Monovalent Mouse IL-12Fc Using PEG-PDBA Polymer Through Protonated Polymer Intermediate

To formulate drug-loaded micelles, the PEG-PDBA polymer was dispersed into methanol at a concentration of 10 mg/mL. 1.05 equivalents of acetic acid per ionizable amine moiety on the polymer was added to protonate and dissolve the polymer. An Amicon Ultra 10k MWCO device was used to remove organic solvent and excess acid from the polymer after protonation by diluting the polymer solution in WFI to the capacity of the Amicon Ultra device, centrifuging, and discarding the permeate. This process was repeated 7 times. The polymer was concentrated to 15 mg/mL at the end of the process. The monovalent mouse IL-12Fc at a concentration of 16.4 mg/mL with the desired quantity (0.1%-20% of polymer weight) was then added to the protonated polymer intermediate from above, mixed gently and incubated overnight with gentle rocking. The mixture was then dialyzed against a neutral buffer, e.g. 10 mM sodium phosphate buffer, at pH 7.4 to complete the encapsulation process.

### Example 3. Encapsulation of a Bivalent Mouse IL-12Fc Using PEG-PDBA Polymer Through Protonated Polymer Intermediate

To formulate drug-loaded micelles, the PEG-PDBA polymer was dispersed into methanol at a concentration of 16 mg/mL. 1.05 equivalents of acetic acid per ionizable amine moiety on the polymer was added to protonate and dissolve the polymer. An Amicon Ultra 10k MWCO device was used to remove organic solvent and excess acid from polymer after protonation by diluting the polymer solution in WFI to the capacity of the Amicon Ultra device, centrifuging, and discarding the permeate. This process was repeated 7 times. The polymer was concentrated to 2.5 mg/mL at the end of this process. The bivalent mouse IL-12Fc at a concentration of 0.125 mg/mL with the desired quantity (0.1%-20% of polymer weight) was then added to the protonated polymer intermediate from above, mixed gently and incubated overnight with gentle rocking. The mixture was then dialyzed against a neutral buffer, e.g. 10 mM sodium phosphate buffer, at pH 7.4 to complete the encapsulation process.

### Example 4. Encapsulation of a Human IL-12 Using PEG-PDBA Polymer Through Protonated Polymer Intermediate

To formulate drug-loaded micelles, the PEG-PDBA polymer was dispersed into methanol at a concentration of 10 mg/mL. 1.05 equivalents of acetic acid per ionizable amine moiety on the polymer was added to protonate and dissolve the polymer. An Amicon Ultra 10k MWCO device was used to remove organic solvent and excess acid from polymer after protonation by diluting the polymer solution in WFI to the capacity of the Amicon Ultra device, centrifuging, and discarding the permeate. This process was repeated 7 times. The polymer was concentrated to 15 mg/mL at the end of this process. The human IL-12 at a concentration of 2 mg/mL with the desired quantity (0.1%-20% of polymer weight) was then added to the protonated polymer intermediate from above, mixed gently and incubated overnight with gentle rocking. The mixture was then dialyzed against a neutral buffer, e.g. 10 mM sodium phosphate buffer, at pH 7.4 to complete the encapsulation process.

### Example 5. Encapsulation of a Single Chain Human IL-12 Using PEG-PDBA Polymer Through Protonated Polymer Intermediate

To formulate drug-loaded micelles, the PEG-PDBA polymer was dispersed into methanol at a concentration of 10 mg/mL. 1.05 equivalents of acetic acid per ionizable amine moiety on the polymer was added to protonate and dissolve the polymer. An Amicon Ultra 10k MWCO device was used to remove organic solvent and excess acid from polymer after protonation by diluting the polymer solution in WFI to the capacity of the Amicon Ultra device, centrifuging, and discarding the permeate. This process was repeated 7 times. The polymer was concentrated to 15 mg/mL at the end of this process. The single chain human IL-12 with the desired quantity (0.1%-20% of polymer weight) was then added to the protonated polymer intermediate from above, mixed gently and incubated overnight with gentle rocking. The mixture was then dialyzed against a neutral buffer, e.g. 10 mM sodium phosphate buffer, at pH 7.4 to complete the encapsulation process.

### Example 6. Encapsulation of a Monovalent Human IL-12Fc Using PEG-PDBA Polymer Through Protonated Polymer Intermediate

To formulate drug-loaded micelles, the PEG-PDBA polymer was dispersed into methanol at a concentration of 10 mg/mL. 1.05 equivalents of acetic acid per ionizable amine moiety on the polymer was added to protonate and dissolve the polymer. An Amicon Ultra 10k MWCO device was used to remove organic solvent and excess acid from polymer after protonation by diluting the polymer solution in WFI to the capacity of the Amicon Ultra device, centrifuging, and discarding the permeate. This process was repeated 7 times. The polymer was concentrated to 15 mg/mL at the end of this process. The monovalent human IL-12Fc with the desired quantity (0.1%-20% of polymer weight) was then added to the protonated polymer intermediate from above, mixed gently and incubated overnight with gentle rocking. The mixture was then dialyzed against a neutral buffer, e.g. 10 mM sodium phosphate buffer, at pH 7.4 to complete the encapsulation process.

### Example 7. Encapsulation of a Bivalent Human IL-12Fc Using PEG-PDBA Polymer Through Protonated Polymer Intermediate

To formulate drug-loaded micelles, the PEG-PDBA polymer was dispersed into methanol at a concentration of 10 mg/mL. 1.05 equivalents of acetic acid per ionizable amine moiety on the polymer was added to protonate and dissolve the polymer. An Amicon Ultra 10k MWCO device was used to remove organic solvent and excess acid from polymer after protonation by diluting the polymer solution in WFI to the capacity of the Amicon Ultra device, centrifuging, and discarding the permeate. This process was repeated 7 times. The polymer was concentrated to 15 mg/mL at the end of this process. The bivalent human IL-12Fc with the desired quantity (0.1%-20% of polymer weight) was then added to the protonated polymer intermediate from above, mixed gently and incubated overnight with gentle rocking. The mixture was then dialyzed against a neutral buffer, e.g. 10 mM sodium phosphate buffer, at pH 7.4 to complete the encapsulation process.

### Example 8. Encapsulation of a Human IL-18 Using PEG-PDBA Polymer Through Protonated Polymer Intermediate

To formulate drug-loaded micelles, the PEG-PDBA polymer was dispersed into methanol at a concentration of 5 mg/mL. 1.05 equivalents of acetic acid per ionizable amine moiety on the polymer was added to protonate and dissolve the polymer. An Amicon Ultra 10k MWCO device was used to remove organic solvent and excess acid from polymer after protonation by diluting the polymer solution in WFI to the capacity of the Amicon Ultra device, centrifuging, and discarding the permeate. This process was repeated 7 times. The polymer was concentrated to 15 mg/mL at the end of this process. The human IL-18 at a concentration of 3.0 mg/mL with the desired quantity (0.1%-20% of polymer weight) was then added to the protonated polymer intermediate from above, mixed gently and incubated overnight with gentle rocking. The mixture was then dialyzed against a neutral buffer, e.g. 10 mM sodium phosphate buffer, at pH 7.4 to complete the encapsulation process.

### Example 9. Encapsulation of a Human IL-2Fc Using PEG-PDBA Polymer Through Protonated Polymer Intermediate

To formulate drug-loaded micelles, the PEG-PDBA polymer was dispersed into methanol at a concentration of 10 mg/mL. 1.05 equivalents of acetic acid per ionizable amine moiety on the polymer was added to protonate and dissolve the polymer. An Amicon Ultra 10k MWCO device was used to remove organic solvent and excess acid from polymer after protonation: this was accomplished by diluting the polymer solution in WFI to the capacity of the Amicon Ultra device, centrifuging, and discarding the permeate. This process was repeated 7 times. The polymer was concentrated to 15 mg/mL at the end of this process. The human IL-2Fc at a concentration of 17.01 mg/mL with the desired quantity (0.1%-20% of polymer weight) was then added to the protonated polymer intermediate from above, mixed gently and incubated overnight with gentle rocking. The mixture was then dialyzed against a neutral buffer, e.g. 10 mM sodium phosphate buffer, at pH 7.4 to complete the encapsulation process.

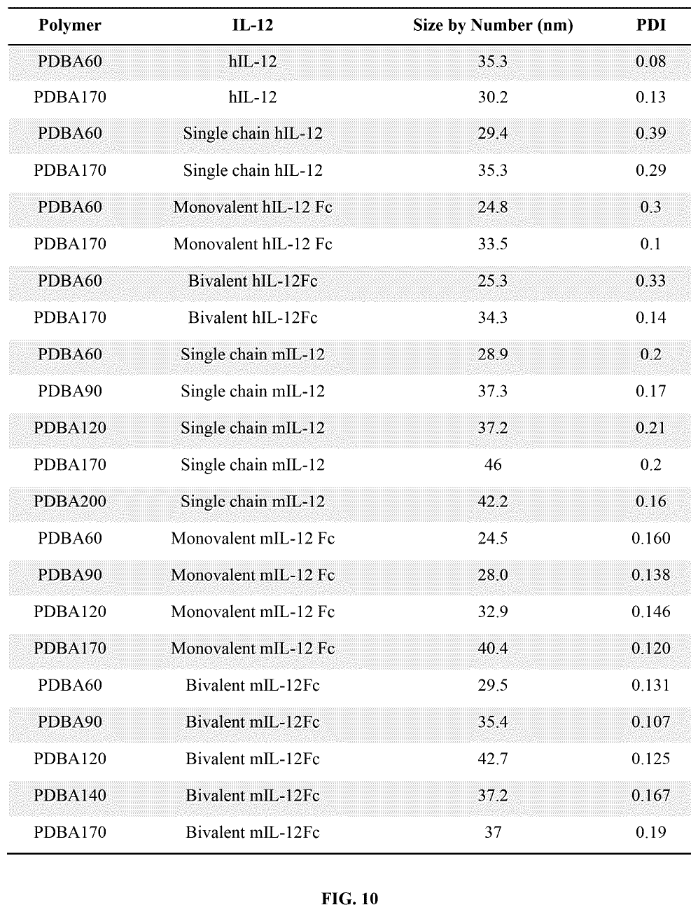

FIG. 10 shows a table of IL-12 encapsulant formulations described in Examples 1-4. The size (nm) and PDI of the encapsulants are reported.

### Example 10. Encapsulation of Solitomab Bispecific Antibody T Cell Engager Using PEG-PDBA Polymer Through Protonated Polymer Intermediate

To formulate drug-loaded micelles, the PEG-PDBA polymer was dispersed into methanol at a concentration of 10 mg/mL. 1.05 equivalents of acetic acid per ionizable amine moiety on the polymer was added to protonate and dissolve the polymer. An Amicon Ultra 10k MWCO device was used to remove organic solvent and excess acid from polymer after protonation by diluting the polymer solution in WFI to the capacity of the Amicon Ultra device, centrifuging, and discarding the permeate. This process was repeated 7 times. The polymer was concentrated at 1.25 mg/mL at the end of this process. The solitomab at a concentration of 0.125 mg/mL with the desired quantity (0.1%-20% of polymer weight) was then added to the protonated polymer intermediate from above, mixed gently and incubated overnight with gentle rocking. The mixture was then dialyzed against a neutral buffer, e.g. 10 mM sodium phosphate buffer, at pH 7.4 to complete the encapsulation process.

### Example 11. Encapsulation of Runimotamab Bispecific Antibody T Cell Engager Using PEG-PDBA Polymer Through Protonated Polymer Intermediate

To formulate drug-loaded micelles, the PEG-PDBA polymer was dispersed into methanol at a concentration of 10 mg/mL. 1.05 equivalents of acetic acid per ionizable amine moiety on the polymer was added to protonate and dissolve the polymer. An Amicon Ultra 10k MWCO device was used to remove organic solvent and excess acid from polymer after protonation by diluting the polymer solution in WFI to the capacity of the Amicon Ultra device, centrifuging, and discarding the permeate. This process was repeated 7 times. The polymer was concentrated to 2.5 mg/mL at the end of this process. The runimotamab at a concentration of 0.125 mg/mL with the desired quantity (0.1%-20% of polymer weight) was then added to the protonated polymer intermediate from above, mixed gently and incubated overnight with gentle rocking. The mixture was then dialyzed against a neutral buffer, e.g. 10 mM sodium phosphate buffer, at pH 7.4 to complete the encapsulation process.

### Example 12. Encapsulation of Blinatumomab Bispecific Antibody T Cell Engager Using PEG-PDBA Polymer Through Protonated Polymer Intermediate

To formulate drug-loaded micelles, the PEG-PDBA polymer was dispersed into methanol at a concentration of 10 mg/mL. 1.05 equivalents of acetic acid per ionizable amine moiety on the polymer was added to protonate and dissolve the polymer. An Amicon Ultra 10k MWCO device was used to remove organic solvent and excess acid from polymer after protonation by diluting the polymer solution in WFI to the capacity of the Amicon Ultra device, centrifuging, and discarding the permeate. This process was repeated 7 times. The polymer was concentrated to 2.5 mg/mL at the end of this process. The blinatumomab at a concentration of 0.125 mg/mL with the desired quantity (0.1%-20% of polymer weight) was then added to the protonated polymer intermediate from above, mixed gently and incubated overnight with gentle rocking. The mixture was then dialyzed against a neutral buffer e.g. 10 mM sodium phosphate buffer at pH 7.4 to complete the encapsulation process.

### Example 13. Encapsulation of Glofitamab Bispecific Antibody T Cell Engager Using PEG-PDBA Polymer Through Protonated Polymer Intermediate

To formulate drug-loaded micelles, the PEG-PDBA polymer was dispersed into methanol at a concentration of 10 mg/mL. 1.05 equivalent of acetic acid per ionizable amine moiety on the polymer was added to protonate and dissolve the polymer. An Amicon Ultra 10k MWCO device was used to remove organic solvent and excess acid from polymer after protonation: this was accomplished by diluting the polymer solution in WFI to the capacity of the Amicon Ultra device, centrifuging, and discarding the permeate. This process was repeated 7 times. The polymer was concentrated to 6.25 mg/mL at the end of this process. The glofitamab at a concentration of 0.125 mg/mL with the desired quantity (0.1%-20% of polymer weight) was then added to the protonated polymer intermediate from above, mixed gently and incubated overnight with gentle rocking. The mixture was then dialyzed against a neutral buffer, e.g. 10 mM sodium phosphate buffer, at pH 7.4 to complete the encapsulation process.

### Example 14. Encapsulation of Odronextamab Bispecific Antibody T Cell Engager Using PEG-PDBA Polymer Through Protonated Polymer Intermediate

To formulate drug-loaded micelles, the PEG-PDBA polymer was dispersed into methanol at a concentration of 10 mg/mL. 1.05 equivalents of acetic acid per ionizable amine moiety on the polymer was added to protonate and dissolve the polymer. An Amicon Ultra 10k MWCO device was used to remove organic solvent and excess acid from polymer after protonation: this was accomplished by diluting the polymer solution in WFI to the capacity of the Amicon Ultra device, centrifuging, and discarding the permeate. This process was repeated 7 times. The polymer was concentrated to 2.5 mg/mL at the end of this process. The odronextamab at a concentration of 0.125 mg/mL with the desired quantity (0.1%-20% of polymer weight) was then added to the protonated polymer intermediate from above, mixed gently and incubated overnight with gentle rocking. The mixture was then dialyzed against a neutral buffer, e.g. 10 mM sodium phosphate buffer, at pH 7.4 to complete the encapsulation process.

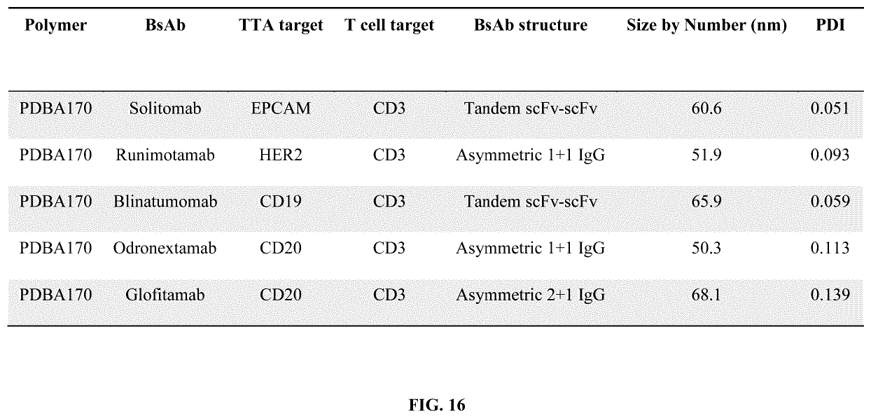

FIG. 16 shows a table characterizing the bispecific encapsulant formulations described in Examples 7-11. The BsAb, TTA target, T cell target, BsAb structure, size by number (nm), and PDI of the encapsulants are reported.

### Example 15. In Vitro Characterization pH-Dependent Activation Window of Mouse or Human IL-12 Formulations by a Reporter Cell Assay

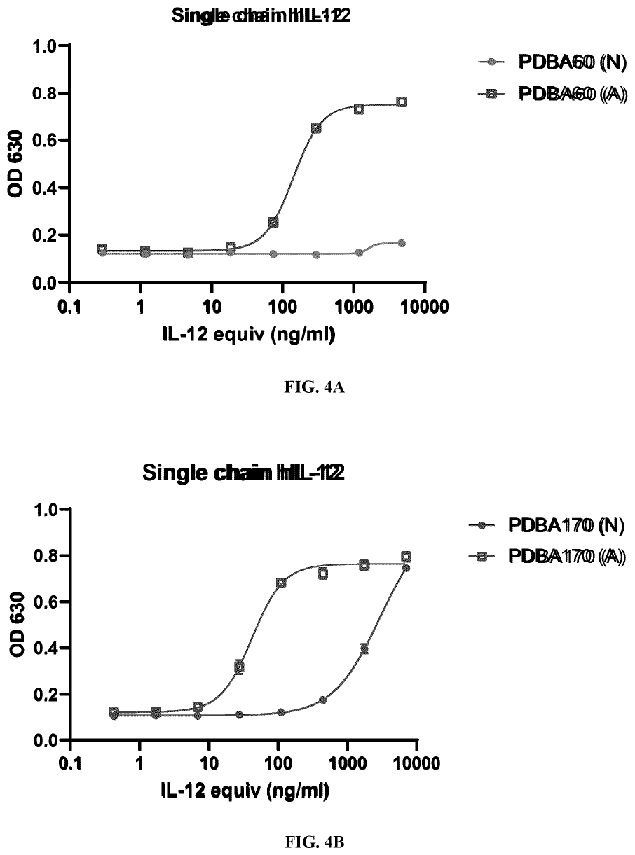

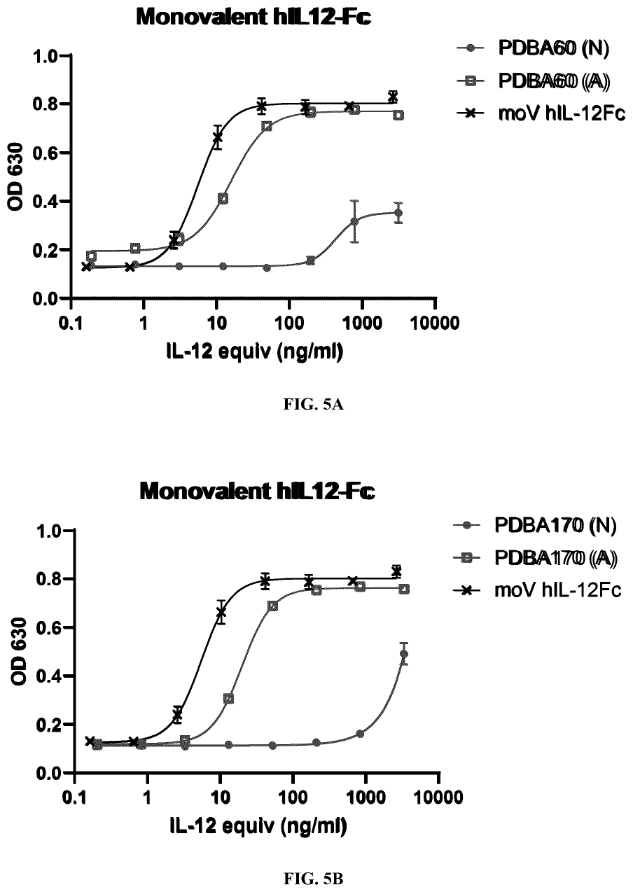

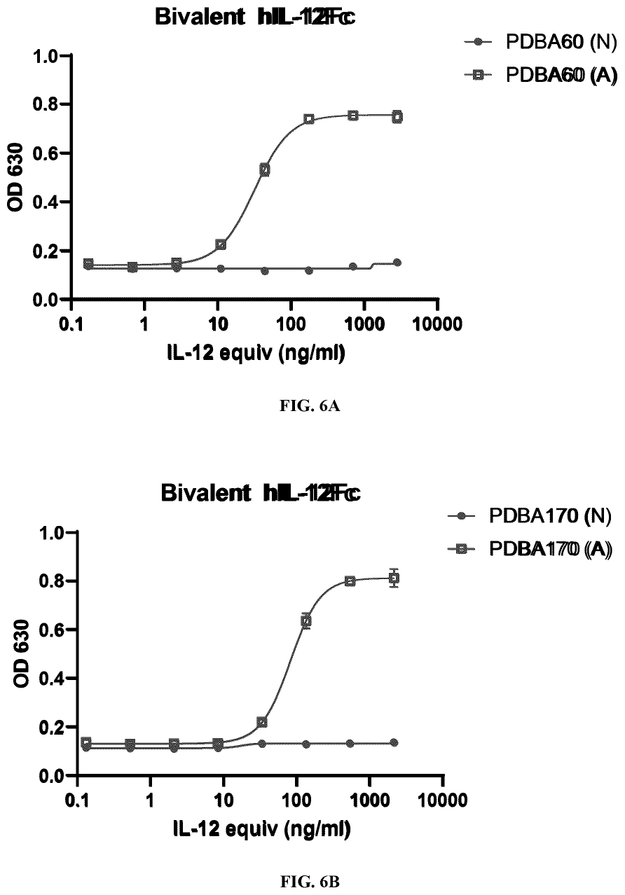

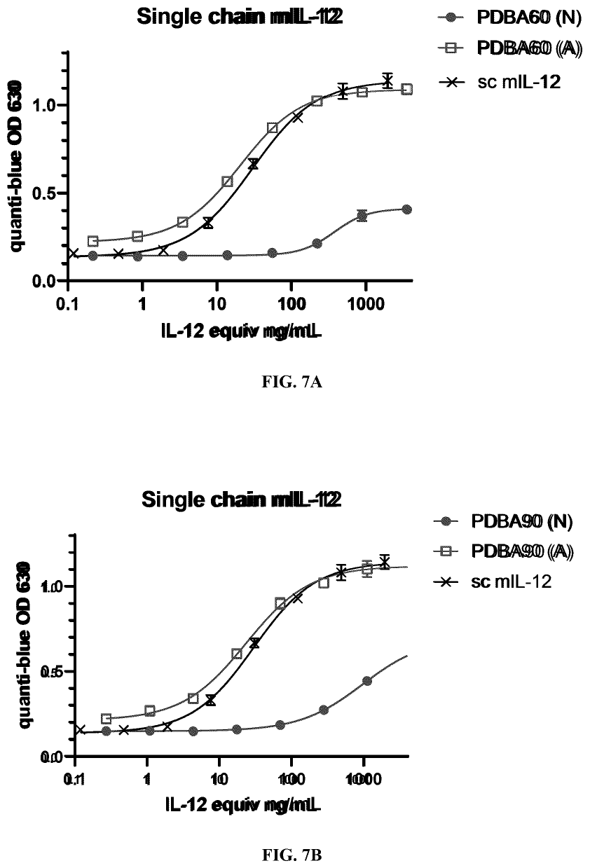

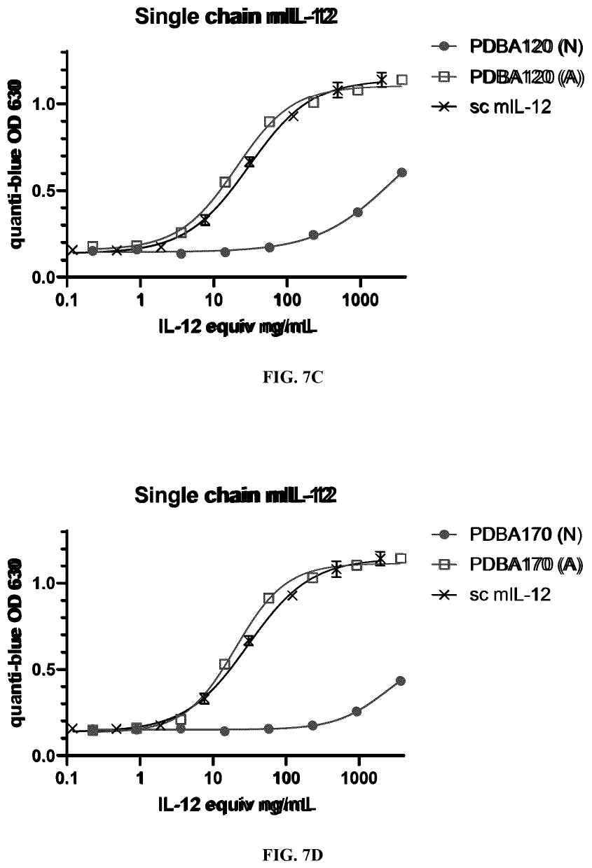

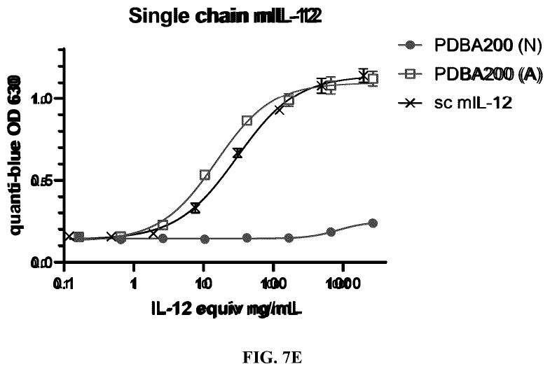

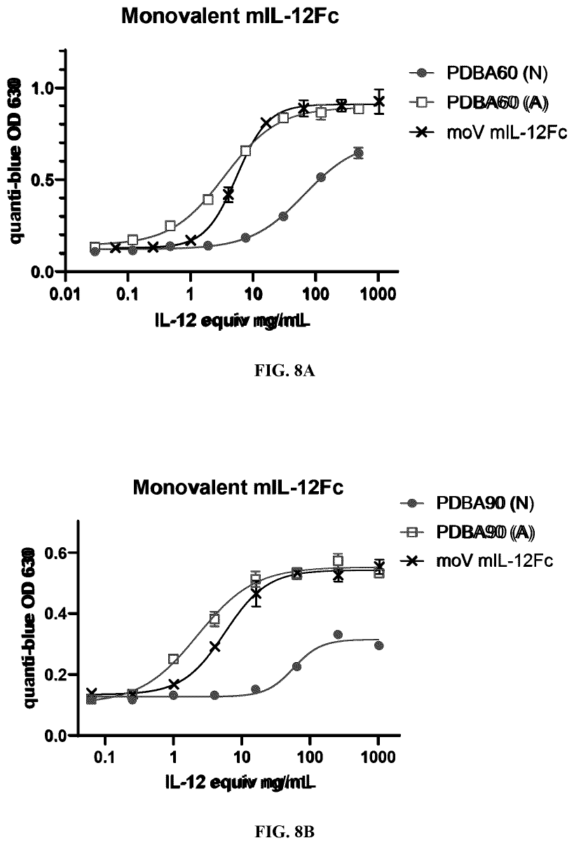

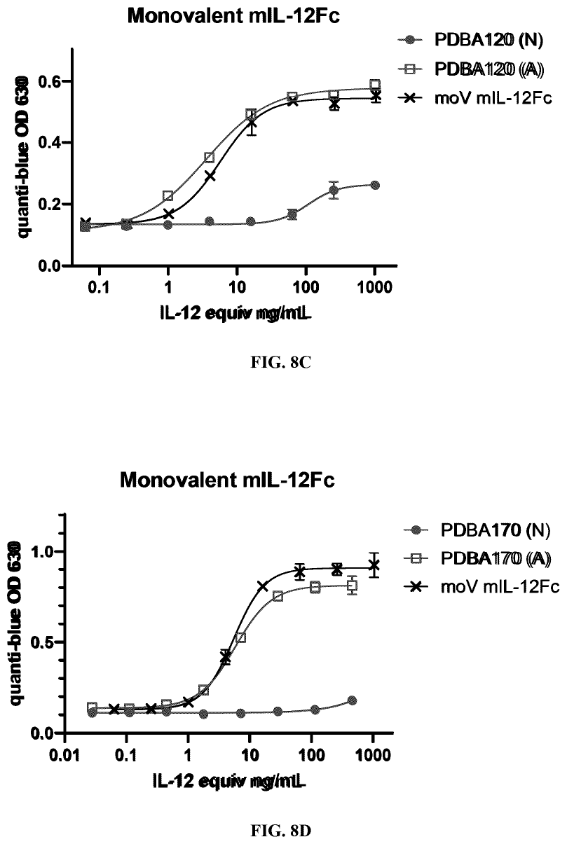

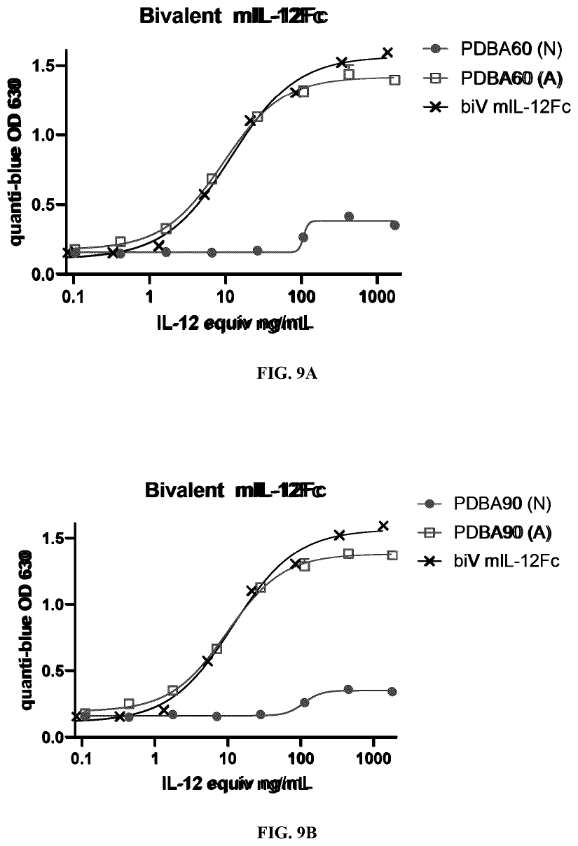

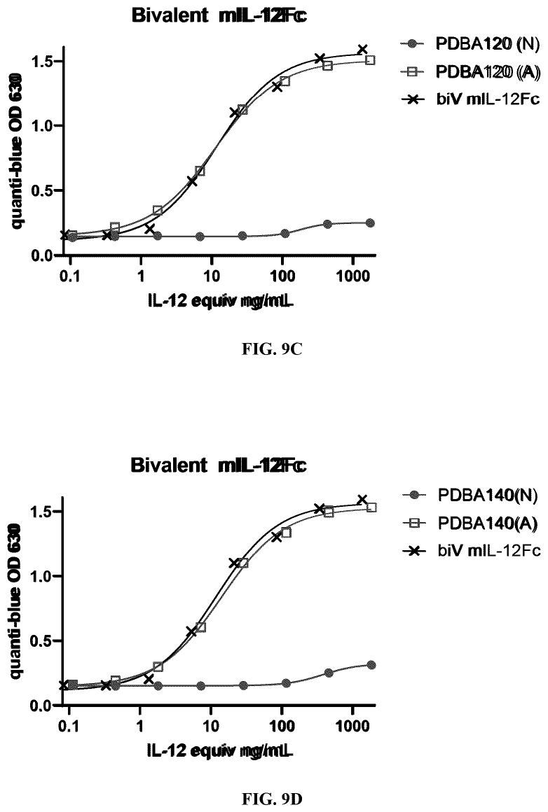

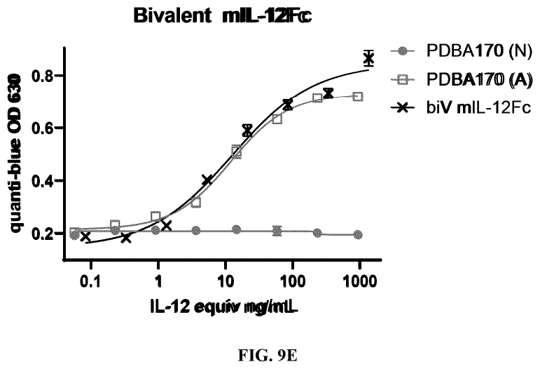

To characterize the pH-dependent activation window, IL-12 formulations were serially diluted in cell culture medium (RPMI1640 10% HI-FBS) and resultant formulation/medium mixture was acidified by adding equal volume of acid cell culture medium to a pH below the pH transition of the formulation. The formulation/medium mixture was then neutralized by adding basic cell culture medium. Proper controls include addition of neutral medium in parallel and the payload proteins as the sample. The above-treated formulations were added to an IL-12 reporter HEK 293 cells. Then IL-12 bioactivity was assessed by adding substrate followed by the measurement of absorbance. (FIG. 2A-B, 3A-B, FIG. 4A-B, FIG. 5A-B, FIG. 6A-B, FIG. 7A-E, FIG. 8A-D, and FIG. 9A-E)

### Example 16: In Vitro Characterization pH-Dependent Activation Window of Human IL-18 Formulations by a Reporter Cell Assay

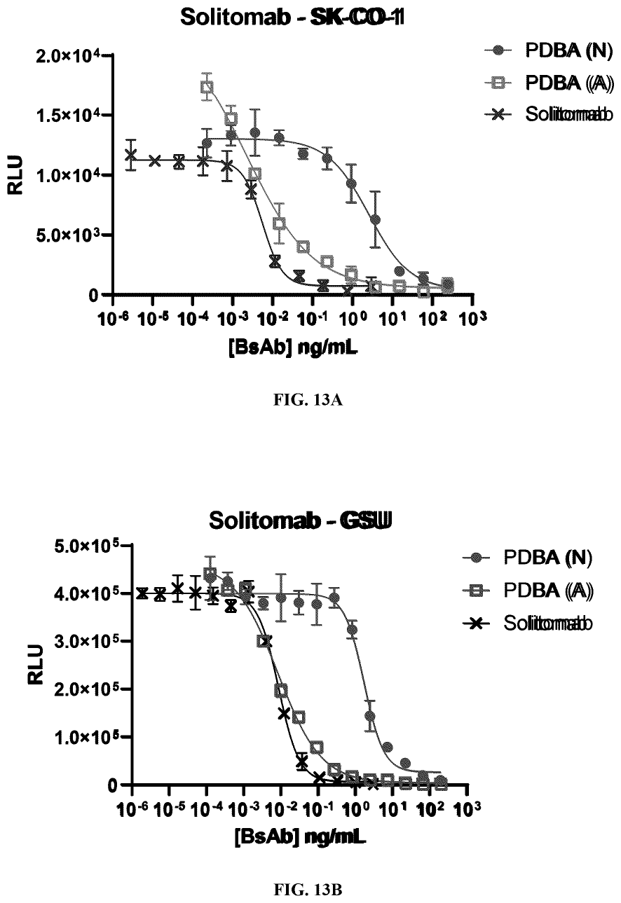

To characterize the pH-dependent activation window, IL-18 formulations were serially diluted in cell culture medium (RPMI1640 10% HI-FBS) and resultant formulation/medium mixture was acidified by adding equal volume of acid cell culture medium to a pH below the pH transition of the formulation. The formulation/medium mixture was then neutralized by adding basic cell culture medium. Proper controls include addition of neutral medium in parallel and the payload proteins as the sample. The above-treated formulations were added to an IL-18 reporter HEK 293 cells. Then IL-18 bioactivity was assessed by adding substrate followed by the measurement of absorbance. (FIG. 12)

### Example 17. In Vitro Characterization pH-Dependent Activation Window of Human IL-2Fc Formulations by a Reporter Cell Assay

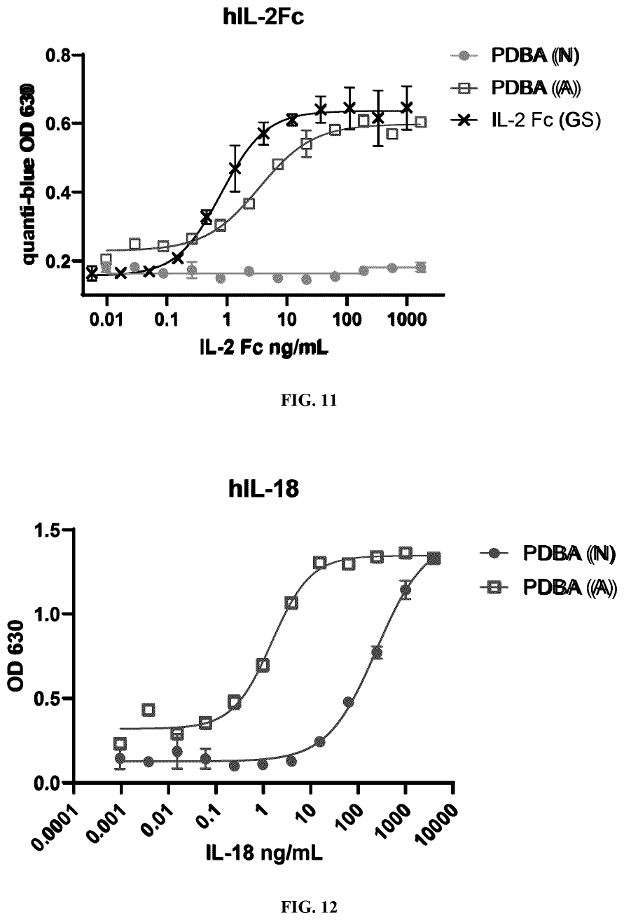

To characterize the pH-dependent activation window, IL-2 formulations were serially diluted in cell culture medium (RPMI1640 10% HI-FBS) and resultant formulation/medium mixture was acidified by adding equal volume of acid cell culture medium to a pH below the pH transition of the formulation. The formulation/medium mixture was then neutralized by adding basic cell culture medium. Proper controls include addition of neutral medium in parallel and the payload proteins as the sample. The above-treated formulations were added to an IL-2 reporter HEK 293 cells. Then IL-2 bioactivity was assessed by adding substrate followed by the measurement of absorbance. (FIG. 2A-B, FIG. 11)

### Example 18. In Vitro Characterization of pH-Dependent Activation Window of Bispecific Antibody T Cell Engager Formulations Using a T Cell Dependent Cellular Cytotoxicity Assay

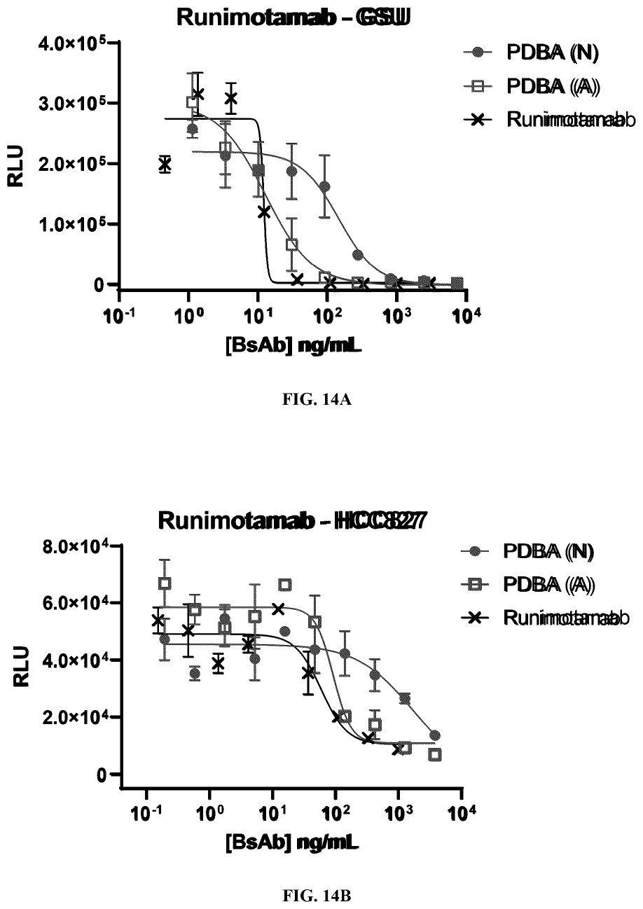

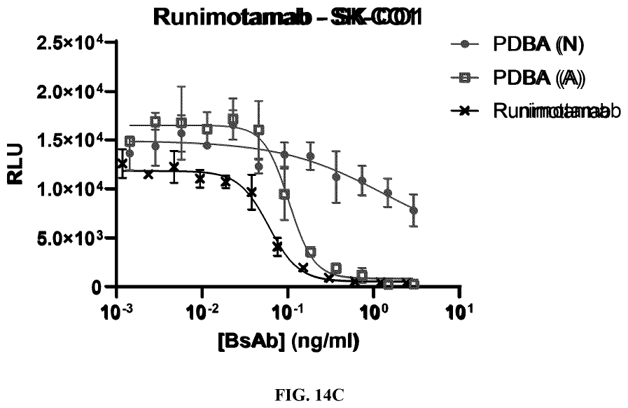

To characterize the pH-dependent activation window, bispecific antibody formulations were serially diluted in cell culture medium (RPMI1640 10% HI-FBS) and the resultant formulation/medium mixture was acidified by adding equal volume of acid cell culture medium to a pH below the pH transition of the formulation. The formulation/medium mixture was then neutralized by adding basic cell culture medium. Proper controls include addition of neutral medium in parallel and the payload proteins as the sample. The above-treated formulations were added to antigen- and firefly luciferase-expressing cancer cell lines and human PBMC (or human pan T cells). The tumor killing ability of bispecific antibodies were assessed by the measurement of bioluminescence from remaining tumor cells after two-day incubation. (FIG. 13A-B, FIG. 14A-C)

### Example 19. In Vitro Characterization of pH-Dependent Activation Window of Bispecific Antibody T Cell Engager Formulations Using a B Cell Depletion Assay

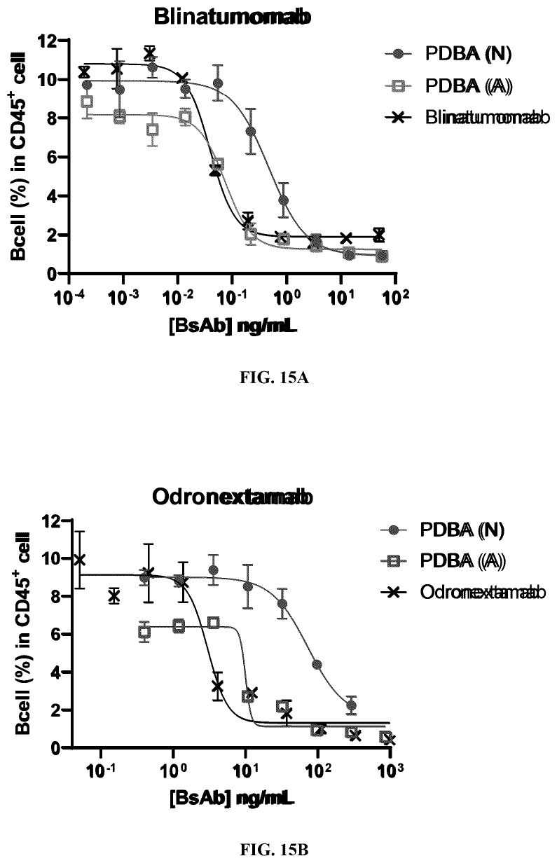

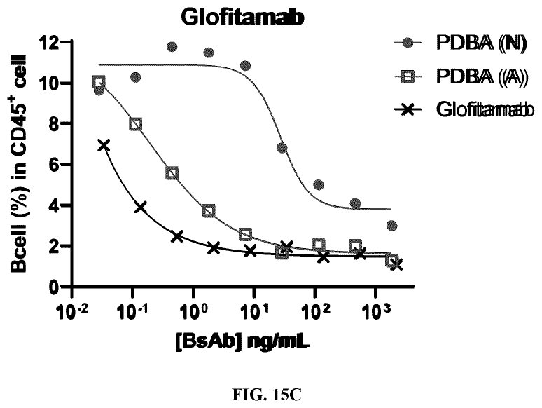

To characterize the pH-dependent activation window, bispecific antibody formulations were serially diluted in cell culture medium (RPMI1640 10% HI-FBS) and the resultant formulation/medium mixture was acidified by adding equal volume of acid cell culture medium to a pH below the pH transition of the formulation. The formulation/medium mixture was then neutralized by adding basic cell culture medium. Proper controls include addition of neutral medium in parallel and the payload proteins as the sample. The above-treated formulations were added to human PBMC (final density 2 million/mL) supplemented with 10 ng/mL recombinant human IL-2. B cell depletion was assessed by the flow cytometry measurement of remaining B cell percentage within CD45+ population after 4-day incubation. B cell was stained by CD19/CD20 antibody. (FIG. 15A-C)

### Example 20. pH-Sensitive Micelle-Encapsulated mIL-12Fc Significantly Reduces Systemic Cytokine Levels, Prevents Liver Toxicity, and Prevents Body Weight Loss

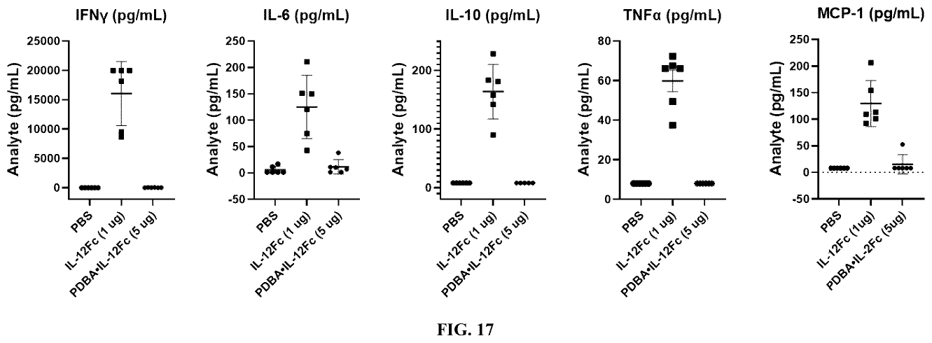

To characterize the systemic cytokine levels of a subject injected with PEG-PDBA pH sensitive micelle encapsulated IL-12Fc, 3 separate groups of healthy BL6 mice were tested. Each group was intravenously administered a control of PBS, 1 μg of free IL12-Fc per injection, or 5 μg PEG-PDBA pH sensitive micelle encapsulated IL-12Fc (PDBA-IL-12Fc) per injection. Injections were administered to the mice on day 0 and day 3 of the study. Plasma samples were taken on day 5 of the study. FIG. 17 illustrates the sampled analyte of the three groups showing significant reduction in IFNγ, IL-6, IL-10, TNFα, and MCP-1 in mice administered the PDBA-IL-12Fc compared to the mice administered with IL-12Fc.

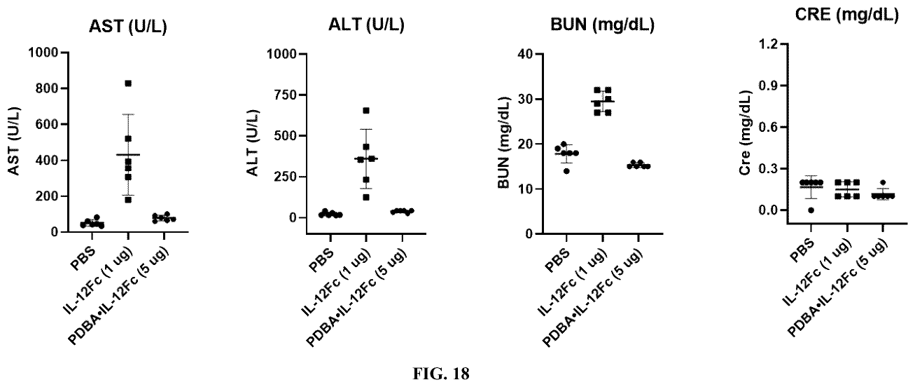

FIG. 18 illustrates the measurements of aspartate transaminase (AST), alanine transaminase (ALT), blood urea nitrogen (BUN), and creatinine (Cre). A significant reduction in AST, ALT, and BUN levels are found in mice treated with PDBA-IL-12Fc as compared to the mice administered with IL-12Fc.

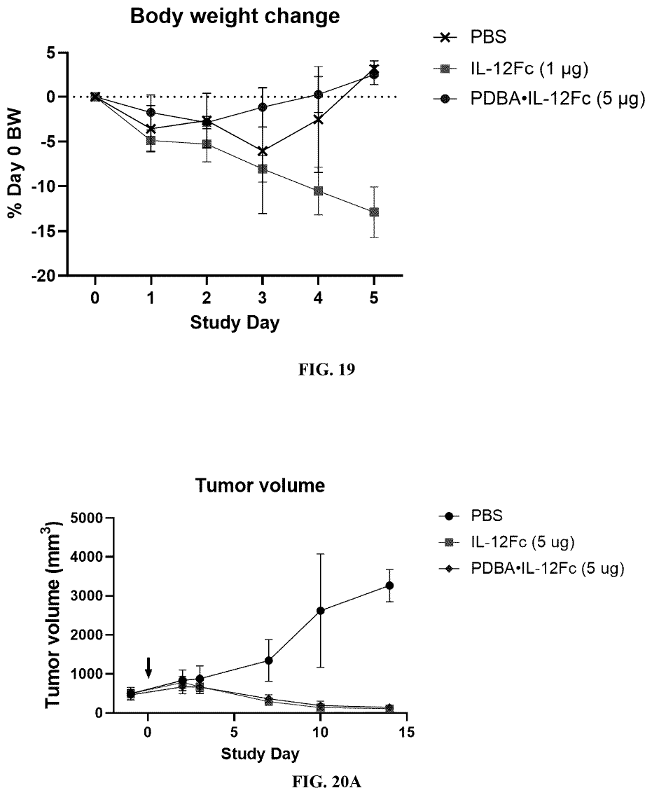

FIG. 19 illustrates the body weight change of the mice of each respective group. The body weight of the mice treated with free IL-12Fc significantly decreased by day 5 of the study whereas the body weight of the mice treated with PDBA-IL-12Fc was relatively unchanged.

### Example 22. Large Tumors (˜500 mm³) Regress Following a Single Injection of pH-Sensitive Micelle Formulation with No Body Weight Loss

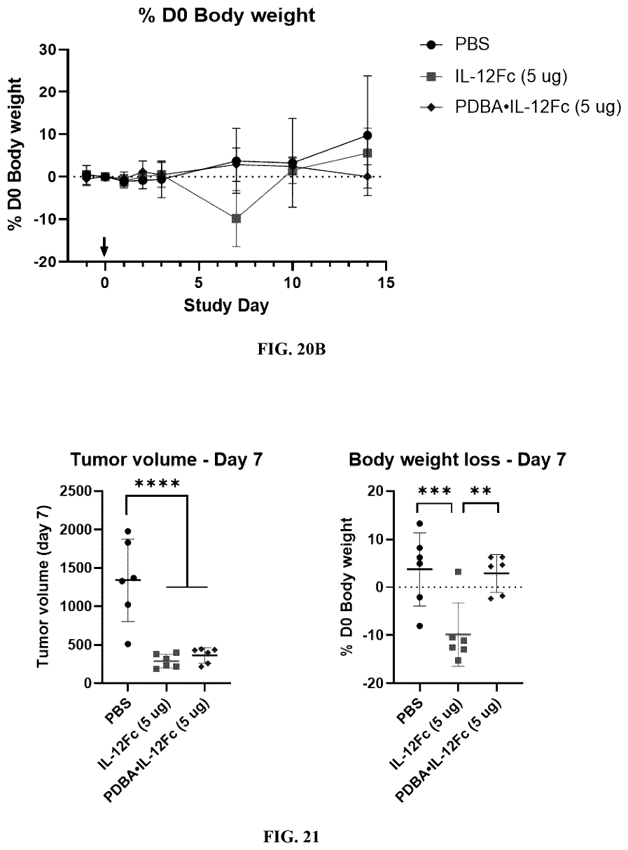

To observe the effects of PEG-PDBA pH sensitive micelle encapsulated IL-12Fc on the size of a large MC38 tumor and the body weight loss of the subject, 3 separate groups of healthy BL6 mice were tested. Each group was intravenously administered a control of PBS, 5 μg of free IL12-Fc, or 5 μg PEG-PDBA pH sensitive micelle encapsulated IL-12Fc (PDBA-IL-12Fc). Injections were administered to the mice as a single injection on day 0 of the study. FIG. 20A illustrates the measurements of tumor size in the mice for each group over the course of the study. Both groups treated with IL-12Fc exhibited reduced tumor sizes while the control group exhibited a growing tumor size. FIG. 20B illustrates the percent change in bodyweight compared to day 0 for each group throughout the course of the study.

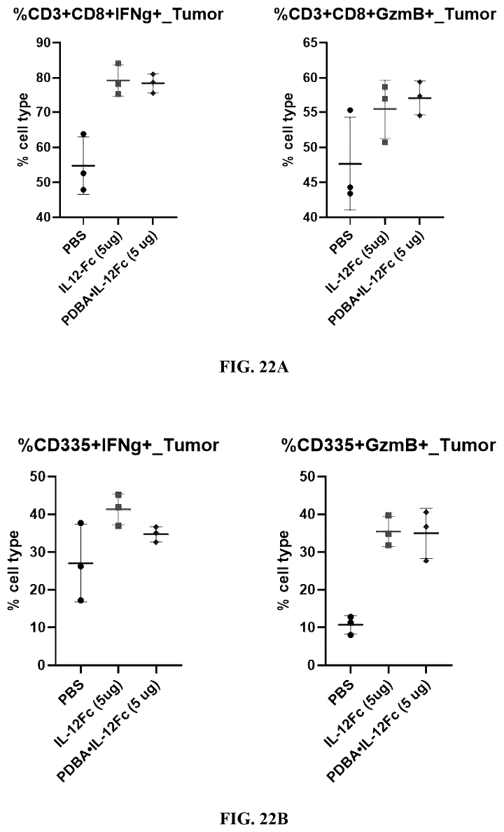

FIG. 21 illustrates the tumor volume and body weight loss percentage of the three groups on day 7 of the study. Both groups treated with the IL-12Fc exhibited comparable tumor volumes which demonstrated tumor regression following the single injection. This shows that the PDBA-IL-12Fc has similar anti-tumor efficacy as free Il-12Fc. However, the group treated with the free IL-12Fc experienced significant weight loss of about 10% as compared to day 0. The control group and the PDBA-IL-12Fc group were comparable with no body weight loss. FIG. 22A and FIG. 22B illustrate an increase in active CD8 T Cells and NK Cells in the tumor of groups treated with both the free IL-12 and the PDBA-IL-12Fc as compared to the control.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method of making an encapsulated biomolecule comprising:
providing an encapsulation composition comprising an organic solvent, a plurality of protonated block copolymer units, and a biomolecule,
encapsulating the biomolecule within a micelle formed from the plurality of protonated block copolymer units,
wherein the block copolymer comprises poly(ethylene oxide) (PEO), and a hydrophobic polymer segment with the following structure: 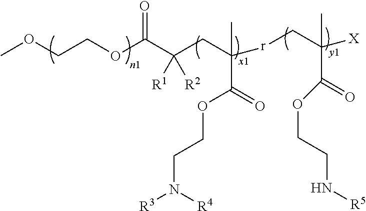
wherein:
n₁ is an integer from about 40 to about 500;
x₁ is an integer from about 4 to about 250;
y₁ is an integer from 0 to about 10;
X is a halogen, —OH, or —C(O)OH;
R¹ and R² are each independently hydrogen or optionally substituted C₁-C₆ alkyl;
R³ and R⁴ are each independently an optionally substituted C₁-C₆ alkyl, C₃-C₁₀ cycloalkyl or aryl;
or R³ and R⁴ are taken together with the corresponding nitrogen to which they are attached form an optionally substituted 5 to 7-membered ring; and
R⁵ is hydrogen or —C(O)CH₃.

2. The method of claim 1, wherein the encapsulation composition is prepared by dissolving the block copolymer in an organic solvent and adding at least a molar equivalent of acid relative to the block copolymer.

3. The method of claim 1, wherein the step of encapsulating the biomolecule within the micelle comprises removing the organic solvent and acid, and adding the therapeutic agent to the polymer.

4. The method of claim 1, wherein the step of encapsulating the biomolecule within the micelle comprises dialyzing a mixture of therapeutic agent and block copolymer against a neutral buffer.

5. The method of claim 1, wherein the hydrophobic polymer segment is selected from:

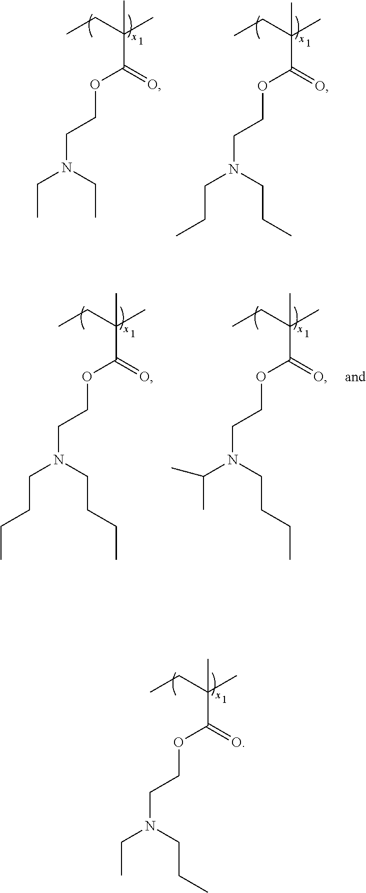

6. The method of claim 5, wherein the hydrophobic polymer segment is selected from:

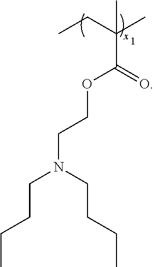

7. The method of claim 1, wherein n₁ is an integer from 100-250, x₁ is an integer from 40-200, and/or y₁ is 0.

8-10. (canceled)

11. The method of claim 1, wherein the step of encapsulating the biomolecule is further comprising of neutralization with a neutral buffer through dialysis or mixing.

12-17. (canceled)

18. The method of claim 1, wherein the biomolecule is a bispecific antibody.

19-43. (canceled)

44. An intermediate composition for making an encapsulated biomolecule comprising:
a biomolecule; and
a plurality of protonated block copolymer units, the block copolymer comprising poly(ethylene oxide) (PEO), and a hydrophobic polymer segment with the following structure: 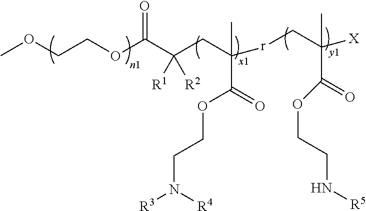
wherein:
n₁ is an integer from about 40 to about 500;
x₁ is an integer from about 4 to about 250;
y₁ is an integer from 0 to about 10;
X is a halogen, —OH, or —C(O)OH;
R¹ and R² are each independently hydrogen or optionally substituted C₁-C₆ alkyl;
R³ and R⁴ are each independently an optionally substituted C₁-C₆ alkyl, C₃-C₁₀ cycloalkyl or aryl;
or R³ and R⁴ are taken together with the corresponding nitrogen to which they are attached form an optionally substituted 5 to 7-membered ring; and
R⁵ is hydrogen or —C(O)CH₃.

45. The intermediate composition of claim 44, wherein the encapsulation composition further comprises an organic solvent and at least a molar equivalent of acid relative to the block copolymer.

46. The intermediate composition of claim 44, wherein the hydrophobic polymer segment is selected from:

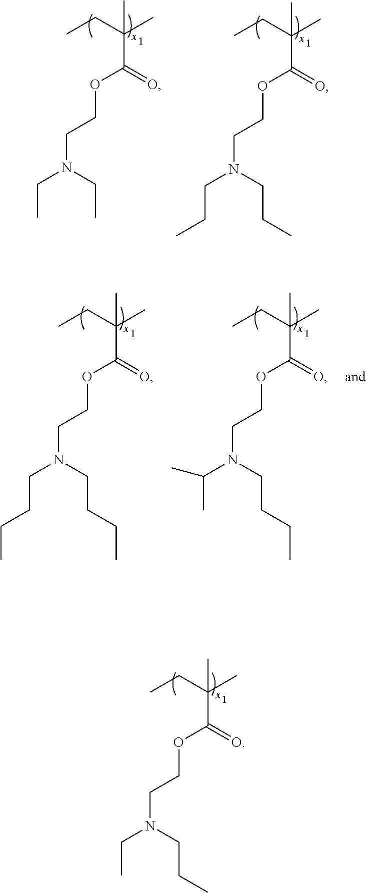

47. The intermediate composition of claim 46, wherein the hydrophobic polymer segment is selected from:

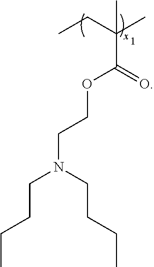

48. The intermediate composition of claim 44, wherein n₁ is an integer from 100-250, x₁ is an integer from 40-200, and/or y₁ is 0.

49-55. (canceled)

56. The intermediate composition of claim 44, wherein the biomolecule is a bispecific antibody.

57-75. (canceled)

76. A micelle encapsulated biomolecule comprising:
a biomolecule; and
a plurality of protonated block copolymer units, the block copolymer comprising poly(ethylene oxide) (PEO), and a hydrophobic polymer segment with the following structure: 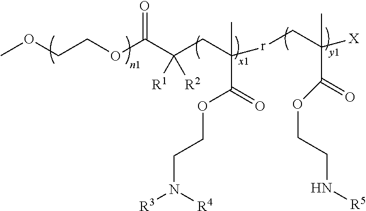
wherein:
n₁ is an integer from about 40 to about 500;
x₁ is an integer from about 4 to about 250;
y₁ is an integer from 0 to about 10;
X is a halogen, —OH, or —C(O)OH;
R¹ and R² are each independently hydrogen or optionally substituted C₁-C₆ alkyl;
R³ and R⁴ are each independently an optionally substituted C₁-C₆ alkyl, C₃-C₁₀ cycloalkyl or aryl;
or R³ and R⁴ are taken together with the corresponding nitrogen to which they are attached form an optionally substituted 5 to 7-membered ring; and
R⁵ is hydrogen or —C(O)CH₃.

77. (canceled)

78. The micelle encapsulated biomolecule of claim 76, wherein the biomolecule is a cytokine or bispecific antibody.

79-80. (canceled)

81. The micelle encapsulated biomolecule of claim 76, wherein the hydrophobic polymer segment is selected from:

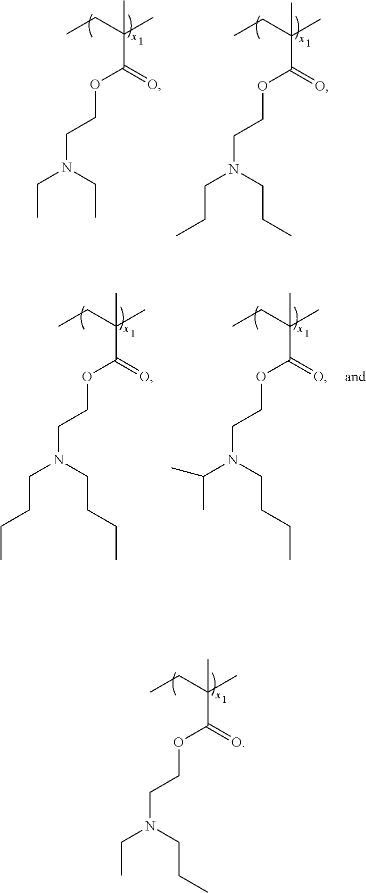

82. The micelle encapsulated biomolecule of claim 76, wherein the hydrophobic polymer segment is selected from:

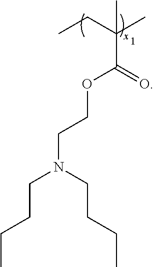

83. The micelle encapsulated biomolecule of claim 76, wherein n₁ is an integer from 100-250, x₁ is an integer from 40-200, and/or y₁ is 0.

84-96. (canceled)
